(19) United States
(12) Patent Application Publication
(10) Pub. No.: US 2024/0376558 A1
(43) Pub. Date: Nov. 14, 2024

(54) COMPOSITIONS AND METHODS FOR SARS CoV-2 RNA DETECTION

(71) Applicant: KING ABDULLAH UNIVERSITY OF SCIENCE AND TECHNOLOGY, Thuwal (SA)

(72) Inventors: Muhammad Shuaib, Thuwal (SA); Arnab Pain, Thuwal (SA)

(21) Appl. No.: 18/559,263

(22) PCT Filed: May 4, 2022
(86) PCT No.: PCT/IB2022/054128

§ 371 Date: Nov. 10, 2022

Related U.S. Application Data
(60) Provisional application No. 63/333,158, filed on Apr. 21, 2022.
(60) Provisional application No. 63/183,933, filed on May 4, 2021.

(51) Int. Cl. C12Q 1/70; C07K 14/005; C12Q 1/6806; C12Q 1/6851

(52) U.S. Cl. CPC C12Q 1/701; C07K 14/005; C12Q 1/6851; C12N 2770/20022; C12Q 1/6806

(57) ABSTRACT

Compositions and methods for enriching, isolating, and/or detecting SARS-CoV-2 RNA in a sample are provided. The compositions include the nucleocapsid (N) protein derived from SARS-CoV-2 virus, or a functional fragment or variant thereof, preferably having one or both of R203K and G204R mutations. The compositions are brought in contact with a sample obtained from a subject. The disclosed compositions can be use in methods of detecting SARS-CoV-2 nucleic acids in a sample such as mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood, thus, methods of detecting SARS-CoV-2 in such samples are also provided.

### CROSS-REFERENCE TO RELATED APPLICATIONS

This application claims priority to and benefit of U.S. Provisional Application No. 63/183,933, filed May 4, 2021, and U.S. Provisional Application No. 63/333,158, filed Apr. 21, 2022, which are incorporated by reference in their entirety.

### FIELD OF THE INVENTION

This invention is generally in the field of compositions and methods for enriching, isolating, and detecting viral RNA, and suited for detecting the presence of SARS-CoV-2 viruses.

### BACKGROUND OF THE INVENTION

The emergence of novel severe acute respiratory syndrome coronavirus 2 (SARS-CoV-2), which causes the respiratory coronavirus infectious disease 2019 (COVID-19), resulted in a pandemic that has triggered an unparalleled public health emergency^{1,2}. The global spread of SARS-CoV-2 depended fundamentally on human mobility patterns and the ability to control human mobility resulted in widespread spread of the virus and its mutant forms.

The development of economical and user-friendly technique for enriching viral RNA will have broad range of applications both in clinical and fundamental research. The isolation of RNA either from patient's samples or infected cells in culture contains both host and viral RNAs. The viral RNA in these samples represents a very tiny fraction and majority of the RNAs are from host cells. The mixture of host and viral RNA is not suitable for many applications; therefore, there is a need for composition and methods of viral RNA enrichment to offer an inexpensive and straightforward tool for viral detection.

It is an object of the present invention to provide compositions and methods for rapid and highly selective enrichment, isolation, and/or detection of SARS-CoV-2 RNA in a biological sample.

It is a further object of the present invention to provide compositions and method for treating a SAR-CoV-2 infection in a subject.

### SUMMARY OF THE INVENTION

Compositions and methods for rapidly and reliably enriching, isolating and/or detecting viral RNAs, preferably SARS-CoV-2 RNAs, within a sample are provided. The disclosed compositions and methods are based on an interaction between the nucleocapsid (N) mutant protein having R203K/G204R mutations and SARS-CoV-2 RNA. Compositions of R203K/G204R N mutant proteins or peptides having at least 70%, 75%, 80%, 85%, 90%, 95%, 96%, 97%, 98%, or 99% sequence identity thereto, and containing the R203K/G204R mutation are provided.

Compositions include capture proteins that typically has a nucleocapsid protein derived from SARS-CoV-2 virus, or a functional fragment or variant thereof, and covalently conjugated thereto one or more capture tags. In some embodiments, the nucleocapsid protein has the amino acid sequence of SEQ ID Nos. 1-4. In preferred embodiments, the nucleocapsid protein has a lysine (K) at the amino acid position 203, or an arginine (R) at the amino acid position 204, or both. In some embodiments, a functional fragment of the nucleocapsid protein includes between about 50 and about 419 contiguous amino acids of the nucleocapsid protein and is sufficient to maintain the biological function of binding to viral RNA. In other embodiments, the variant of the nucleocapsid protein has at least 65%, 70%, 75%, 80%, 85%, 90%, 95%, 99% or 100% sequence identity to SEQ ID NOs:1, 2, 3, or 4. Capture tags are typically covalently bound to the nucleoprotein. Exemplary capture tags include hexa-histidine tag, FLAG tag, Strep II tag, streptavidin-binding peptide (SBP) tag, calmodulin-binding peptide (CBP), glutathione S-transferase (GST), maltose-binding protein (MBP), S-tag, the hemagglutinin (HA) tag, and c-Myc tag.

Capture proteins can be immobilized to a support matrix. In some embodiments, the support matrix is a solid phase selected from the group consisting of glass plates, microtiter well plates, magnetic beads, and silicon wafers. In preferred embodiments, the solid phase is magnetic beads, such as spherical beads having a diameter of about 1 μm to about 10 μm, inclusive, or about 2 μm and about 8 μm, inclusive, or about 3 μm and about 6 μm.

Complexes including RNAs specifically bound to capture proteins are also provided. In some embodiments, the RNAs are viral RNAs derived from an RNA virus, such as a coronavirus. In preferred embodiments, the RNAs are viral RNAs derived from SARS-CoV-2.

Kits including the capture protein, preferably immobilized to a solid phrase such as magnetic beads and/or affinity-binding column, optionally one or more wash buffers, elution buffers, and instructions, are also provided. In some embodiments, kits include buffers and reagents required RT-qPCR amplification of SARS-CoV-2 viral RNA.

Methods of enriching and/or isolating viral RNAs are also provided. Methods typically include the step of (a) contacting a sample of interest with the capture protein, preferably immobilized to a solid phase such as magnetic beads, in a suitable binding buffer. Exemplary solid phase includes glass plates, microtiter well plates, magnetic beads, and silicon wafers. Suitable binding buffer has a pH of about 7 to about 8, preferably 7.5, and further preferably with one or more RNase inhibitors. In some embodiments, the methods further include the step of (b) removing supernatant after step (a), optionally (c) one or more washing steps to remove unbound and/or non-specific bound molecules. In preferred embodiments, the methods further include (d) isolating the capture protein complexed with viral RNA, and/or (e) eluting isolated viral RNA from the capture protein using an appropriate buffer. In some embodiments, the methods also involve (f) quantitating the isolated viral RNA, for example via amplifying one or more genomic segments of the isolated viral RNA. In some case, transcription quantitative polymerase chain reaction (RT-qPCR) is used to quantitate the isolated viral RNA. In some embodiments, the isolated RNAs are viral RNAs derived from an RNA virus, such as a coronavirus. In preferred embodiments, the RNAs are viral RNAs derived from SARS-CoV-2.

Samples of interest used in the disclosed methods can be an environmental sample or a biological sample. In some embodiments, the biological sample is a bodily fluid of a subject, the bodily fluid selected from the group consisting of mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, and whole blood. Thus, in some embodiments, the methods further include a step of obtaining the biological sample from the subject, for example obtaining from a nasopharyngeal swab, a nasopharyngeal aspirate, sputa/deep throat saliva, or a throat swab of the subject. In preferred embodiments, the subject has one or more symptoms of COVID-19. In other embodiments, the subject is an asymptomatic subject who is at increased risk of being infected with SARS-CoV-2 virus, a subject who has received a vaccine against infection with SARS-CoV-2 virus, or a deceased subject. Typically the sample of interest contains 10 or more copies of the SARS-CoV-2 genomes.

### BRIEF DESCRIPTION OF THE FIGURES

FIG. 1A. Locations of the sampling cities within Saudi Arabia. FIG. 1B. Combined numbers of samples retrieved from the 4 cities and the Eastern region during the first six months of the pandemic. Cities are colored as in panel A. Months are shown at the bottom of the figure, and each month is divided into 5-days intervals. New daily cases for the city of Khobar are shown on the Eastern Region plot. Major restrictions imposed by the Ministry of Health and by Royal decrees are indicated above plots. FIG. 1C. Combined average numbers of new daily cases. FIG. 1D. Estimate of effective reproduction number [R_{eff}] over time in Saudi Arabia (top) and the estimate of effective population size [Ne], the relative population size required to produce the diversity seen in the sample (bottom). The red horizontal red line represents an R of 1, the level required to sustain epidemic growth. Grey confidence areas denote the 95% credible intervals. FIG. 1E. The 836 detected SNPs are shown along their positions in the SARS-CoV-2 genome (x-axis) and their frequency in the Saudi samples (y-axis). High-frequency SNPs are highlighted along with the 3 SNPs underlying the R203K/G204R changes in the N protein (G28881A; G2882A; and G28883C)

FIG. 2 Distributions of importation dates for the 5 Nextstrain (nextstrain.org) clades found in Saudi Arabia coloured by clade.

FIG. 3A-G. SNP analysis. FIG. 3A. Manhattan plot showing the association between SARS-CoV-2 SNPs and recorded mortality. Negative log₁₀(uncorrected p-values) from Fisher's exact tests are shown as red circles. Gene boundaries are indicated by background colors (listed on top), and the three R203K/G204R SNPs (positions 28,881-28,883) in the N gene are highlighted. FIG. 3B. Overview of the three SNPs underlying the R203K/G204R changes. Amino acid numbers in the N protein is shown above. FIG. 3C. The number of samples with and without the R203K/G204R SNPs and their recorded mortality rates. Odds ratio is calculated as (diseased with SNPs/diseased without SNPs)/(not diseased with SNPs/not diseased without SNPs). FIG. 3D. Samples with and without the R203K/G204R SNPs shown as bars (left y-axis) for different patient age groups (x-axis). The mortality rates for each age group are shown as lines (right x-axis). FIG. 3E. The numbers of Saudi samples with (dark bars) and without (light bars) the R203K/G204R SNPs are shown through 2020. The R203K/G204R frequencies are shown as circles connected by lines (right y-axis). FIG. 3F. Similar to panel FIG. 3E) but showing numbers for non-Saudi samples deposited in GISAID. FIG. 3G. The number of samples with R203K/G204R and reference alleles in 1,419 samples with patient status metadata available in GISAID divided into mild (825 samples) and severe cases (594 samples). R203K/G204R SNP frequencies are listed above bars, and p-value from Fisher's exact two-sided test is shown above plot.

FIG. 4A-C. Identification of host-interacting partners and profiling of phosphorylation status of mutant and control SARS-CoV-2 N protein by Affinity Mass-Spectrometry. FIG. 4A. A schematic diagram showing the SARS-CoV-2 N protein different domains (Upper: control, Lower: mutant) and highlighting the mutation site (R203K and G204R). The bar-plot (lower panel) indicates the SIFT²¹ predicted deleteriousness score of substitution at position 204 from G to R. FIG. 4B. Sketch showing part of SR-rich motif of SARS-CoV-2 N protein containing the KR mutation site (R203K and G204R) (Lower). The hyper-phosphorylated serine 206 in the mutant N protein near the KR mutation site is indicated in orange color. FIG. 4C. Phosphorylation status of mutant and control N protein was analyzed by mass spectrometry (3 biological replicates per affinity condition). Bar-plot shows the Log 2 intensities of selected phosphorylated peptides in control and mutant condition. Serine 206 is hyper-phosphorylated in mutant N protein.

FIG. 5A-5B show Co-occurrences of SNPs shown as Jaccard Index (FIG. 5A) and log 2 odds-ratio (FIG. 5B). The co-occurrence between the three SNPs in the R203K & G204R mutations (as indicated above plots) and all SNPs present in at least 20 samples (x-axes) are shown as circles. Co-occurrences between the three SNPs are highlighted in orange

FIG. 6 is a box plot showing the distribution of virus copy number derived from Ct measurements. Copy numbers are shown for four different haplotypes (as indicated below the plot) corresponding to virus genome positions 28,881-28,883 (N protein) and 23,403 (Spike protein). ‘Wuhan’ denotes the genotypes in the reference genome (NC_045512).

FIG. 7A-D. Oligomerization and RNA binding analysis of mutant and control N protein. FIG. 7A. BS3 cross-linking (2 mM) and SDS-PAGE analysis of the oligomerization forms of mutant and control N proteins. FIG. 7B. Densitometry analysis of bands corresponding to oligomeric forms (trimer and tetramer) was performed. Bar-plot represents the relative intensities from two independent experiments (as shown mean±SD). FIG. 7C. Sketch of in vitro RNA immunoprecipitation (RIP) procedure used for analysis of viral RNA interaction with purified mutant and control N protein. Isolated RNAs were analyzed by RT-qPCR using specific primers for viral N gene (N1 and N2) and E gene. FIG. 7D. Bar chart shows level of viral RNA retrieval (% input) with mutant and control N protein (+SD from 4 experiments, [t-test, p value 0.00016 (***), 0.00019 (***), and 0.003 (**)]).

FIG. 8A-E. Affinity mass spectrometry (AP-MS) analysis of mutant and control SARS-CoV-2 N protein and host protein interaction. FIG. 8A. An outline showing the workflow of affinity mass spectrometry procedure. HEK-293 cell expressing 2λStrep-tagged control and mutant N protein were used for MagStrep affinity purification. Purified proteins were separated on SDS-PAGE and subjected to silver staining and western blotting for confirmation. After confirmation, interacting proteins were analyzed on mass spectrometry. FIG. 8B. (Upper) Silver staining of control and mutant N protein associated host proteins (1 &2 show two loading volume). (Lower) Western blot confirmation of N protein (mutant and control) using anti-Strep antibody. FIG. 8C. Correlation matrix of three replicates for control and mutant N protein AP-MS. FIG. 8D. The amount of total N protein detected by mass spectrometry in the control and mutant affinity. Graph shows log 2 of protein intensities. FIG. 8E. Level of stress granule proteins (G3BP1 and G3BP2) identified in control and mutant condition.

### DETAILED DESCRIPTION OF THE INVENTION

The disclosed compositions and methods are based on studies in which 892 SARS-CoV-2 genomes collected from patients in Saudi Arabia from March to August 2020 were sequenced. The studies (as well as global data analysis) showed a clear association between patient mortality and two consecutive mutations (R203K/G204R) in the SARS-CoV-2 nucleoprotein (N). These mutations affect the oligomerization of N protein and its binding to viral RNA, as well as its interaction with host proteins. Furthermore, the mutations result in the phosphorylation of a nearby serine site (S206) in the N protein.

Compositions for enrichment of viral RNA are disclosed. In the research projects where viral RNA enrichment is required mostly depend on biotin-labeled nucleic acid probes that must cover the whole viral genome. However, there are many limitations: (i) nucleic acid probes are expensive; (ii) nucleic acid probes lack optimal efficacy in the case of viruses that undergo continuous mutations, such as SARS-CoV-2; (iii) multiple probes are required to enrich the complete viral RNA genome; and (iv) the protocol used for probe-based enrichment is tedious and requires highly skilled, experienced practitioners. The disclosed compositions and methods for N protein based viral RNA enrichment provide enhanced identification of viral RNA in biological samples, with high resolution. Kits for the detection, enrichment and isolation of SARS-CoV-2 RNA are provided.

### I. Definitions

The terms “Isolated,” “isolating,” “purified,” “purifying,” “enriched,” and “enriching,” when used with respect to nucleic acids of interest, indicate that the nucleic acids of interest at some point in time are separated from or with respect to a mixture containing other cellular material. Typically, the isolating yields a higher proportion of the nucleic acids of interest compared to the other cellular material, which can include contaminants, including active agents such as enzymes. “Highly purified,” “highly enriched,” and “highly isolated,” when used with respect to nucleic acids of interest, indicates that the nucleic acids of interest are at least about 70%, about 75%, about 80%, about 85%, about 90% or more, about 95%, about 99% or 99.9% or more purified or isolated from the other cellular materials, contaminants, or active agents such as enzymes. “Substantially isolated,” “substantially purified,” and “substantially enriched,” when used with respect to nucleic acids of interest, indicate that the nucleic acids of interest are at least about 70%, about 75%, or about 80%, more usually at least 85% or 90%, and sometimes at least 95% or more, for example, 95%, 96%, and up to 100% purified or isolated from other cellular materials, contaminants, or active agents such as enzymes.

The terms “SARS-CoV-2” and “Severe Acute Respiratory Syndrome Coronavirus 2” refer to the pathogenic coronavirus strains of the subgenus Sarbecovirus which are derived from the betacoronavirus of zoonotic origin which emerged in Asia in late 2019, and which are the causative agents of pandemic Coronavirus disease 2019 (COVID-19) in humans. SARS-CoV-2 have a high rate of genetic mutation in its genome, resulting in variants. Multiple variants of the virus that causes COVID-19 have been documented globally during this pandemic including a variant called B.1.1.7 identified in the United Kingdom, a variant called B.1.351 identified in South Africa, and a variant called P.1 identified in Brazil.

The term “N gene” refers to the viral gene which encodes the nucleocapsid protein, located at the 3′ region of the SARS-CoV-2 coronavirus RNA genome encoding a polyprotein. A representative N gene from the SARS-CoV-2 coronavirus is deposited in GenBank as accession No: MN908947.3.

The term “conditions sufficient for” as used herein in connection with the disclosed methods, refers to any environment that permits the desired activity, for example, that permits specific binding between nucleocapsid (N) protein derived from SARS-CoV-2 virus, or a fragment thereof, and viral RNAs. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), etc).

The term “contact” as used herein in connection with the disclosed methods refers to placement in direct physical association; for example, in solid and/or liquid form. For example, contacting can occur in vitro with one or more primers and/or probes and a biological sample (such as a sample including nucleic acids) in solution.

The terms “subject,” “individual” or “patient” refer to a human or a non-human mammal. A subject may be a non-human primate, domestic animal, farm animal, or a laboratory animal. For example, the subject may be a dog, cat, goat, horse, pig, mouse, rabbit, or the like. The subject may be a human. The subject may be healthy or suffering from or susceptible to a disease, disorder, or condition. A patient refers to a subject afflicted with a disease or disorder. The term “patient” includes human and veterinary subjects.

A “control” sample or value refers to a sample that serves as a reference, usually a known reference, for comparison to a test sample. For example, a test sample can be taken from a test subject, and a control sample can be taken from a control subject, such as from a known normal (non-disease) individual. A control can also represent an average value gathered from a population of similar individuals, e.g., disease patients or healthy individuals with a similar medical background, same age, weight, etc. One of skill will recognize that controls can be designed for assessment of any number of parameters.

The term “specificity” refers to the ability of a test to correctly identify true negatives, i.e., samples that have no SARS-CoV-2 infection. For example, specificity can be expressed as a percentage, the proportion of actual negatives which are correctly identified as such (e.g., the percentage of test samples not having SARS CoV-2 correctly identified by the test as not having SARS-CoV-2). A test with high specificity has a low rate of false positives, i.e., the cases of samples not having SARS-CoV-2 but suggested by the test as having SARS-CoV-2. Therefore, a specificity of 90% indicates a total 10% of false positives. Generally, the disclosed methods have a specificity of at least 90%, at least 92%, at least 94%, at least 95%, at least 96%, at least 97%, at least 98%, at least 99%, or at least 100%.

The term “screening” refers to testing a sample, such as a biological sample from an individual, or from a population of individuals, with known or unknown status of infection.

The terms “detect,” and “identify,” in the context of an assay are used interchangeably and refer to the positive identification of a target, such as genetic component of a coronavirus. The identification or detection can be interpreted or assessed according to the mechanism of an assay, and identification or detection can be compared to a control or to a standard level. For example, in a RT-qPCR assay, the extent of detection of a gene or expressed gene product may be quantified as complete (i.e., 100%) or partial (i.e., 1-99.9%) of the expected or calculated level of that in a control. Quantitation can be measured as a % value, e.g., from 1% up to 100%, such as 5%, 10, 25, 50, 75, 80, 85, 90, 95, 99, or 100%. For example, the relative amount of a target gene, or the activity or quantity of one or more expressed gene products can be assessed relative to a control, or relative to another experimental sample. In some embodiments, the detection or quantitation are compared according to the level of RNAs, or proteins corresponding to the targeted genetic element within a control cell.

The term “inhibit” or other forms of the word such as “inhibiting” or “inhibition” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” can also mean to hinder or restrain the synthesis, expression or function of a protein relative to a standard or control. Inhibition can include, but is not limited to, the complete ablation of the activity, response, condition, or disease. “Inhibits” can also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels. For example, “inhibits expression” means hindering, interfering with, or restraining the expression and/or activity of the gene/gene product pathway relative to a standard or a control.

“Treatment” or “treating” means to administer a composition to a subject or a system with an undesired condition (e.g., COVID-19). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization, or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. “Prevention” or “preventing” means to administer a composition to a subject or a system at risk for an undesired condition (e.g., COVID-19). The condition can include one or more symptoms of a disease, pathological state, or disorder. The condition can also be a predisposition to the disease, pathological state, or disorder. The effect of the administration of the composition to the subject can be the cessation of a particular symptom of a condition, a reduction or prevention of the symptoms of a condition, a reduction in the severity of the condition, the complete ablation of the condition, a stabilization or delay of the development or progression of a particular event or characteristic, or reduction of the chances that a particular event or characteristic will occur.

The terms “effective amount” or “therapeutically effective amount” means a quantity sufficient to alleviate or ameliorate one or more symptoms of a disorder, disease, or condition being treated, or to otherwise provide a desired pharmacologic and/or physiological effect. Such amelioration only requires a reduction or alteration, not necessarily elimination. The precise quantity will vary according to a variety of factors such as subject-dependent variables (e.g., age, immune system health, weight, etc.), the disease or disorder being treated, as well as the route of administration, and the pharmacokinetics and pharmacodynamics of the agent being administered.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

The term “polypeptides” includes proteins and functional fragments thereof. Polypeptides are disclosed herein as amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term “functional fragment” or “functional variant” means a fragment or variant of a polypeptide, such as a full-length or native polypeptide, that retains one or more functional properties of the full-length or native polypeptide. For example, in some embodiments, a functional fragment or functional variant of a nucleoprotein is a fragment or variant that retains the function of binding to viral RNA.

The terms “variant” or “active variant” refers to a polypeptide or polynucleotide that differs from a reference polypeptide or polynucleotide, but retains one or more functional properties (e.g., functional or biological activity). A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally. Modifications and changes can be made in the structure of the polypeptides of the disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological or functional activity, certain amino acid sequence substitutions can be made in a polypeptide sequence and nevertheless obtain a polypeptide with like properties (e.g., functional or biological activity).

Modifications and changes can be made in the structure of the polypeptides of in disclosure and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity, particularly, where the biological functional equivalent polypeptide or peptide thereby created is intended for use in immunological embodiments. The following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure thus contemplate functional or biological equivalents of a polypeptide as set forth above. In particular, embodiments of the polypeptides can include variants having about 50%, 60%, 70%, 80%, 90%, and 95% sequence identity to the polypeptide of interest.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” can also mean the degree of sequence relatedness of a polypeptide compared to the full-length of a reference polypeptide. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M, and Griffin, H. G., Eds., Humana Press, New Jersey, 1994; Sequence Analysis in Molecular Biology, von Heinje, G., Academic Press, 1987; and Sequence Analysis Primer, Gribskov, M and Devereux, J., Eds., M Stockton Press, New York, 1991; and Carillo, H, and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other aspects the values may range in value either above or below the stated value in a range of approx. +/−5%; in other aspects the values may range in value either above or below the stated value in a range of approx. +/−2%; in other aspects the values may range in value either above or below the stated value in a range of approx. +/−1%.

### II. Compositions

Compositions that rapidly and reliably enrich, isolate and/or detect viral RNAs, preferably SARS-CoV-2 RNAs, within a sample have been established. The compositions employ one or more labelled capture proteins that specifically bind to a target viral RNA with high affinity.

### A. Capture Proteins

Capture proteins that bind to a viral target include one or more specific labels or capture tags are described.

### 1. Nucleocapsid Proteins

A preferred capture protein is a nucleocapsid protein with an R203K/G204R mutation relative to SEQ ID NO:1. A preferred nucleocapsid is the (N) protein derived from SARS-CoV-2 virus, or a functional fragment or variant thereof.

A representative N gene from the SARS-CoV-2 coronavirus is deposited in GenBank as accession No: MN908947.3. An exemplary amino acid sequence for the N protein (GenBank accession No. QHD43423) is set forth below SE ID No. 1):

| | |
|---|---|
| 1 | MSDNGPQNOR NAPRITFGGP SDSTGSNQNG ERSGARSKQR RPQGLPNNTA |
| 51 | SWFTALTQHG KEDLKFPRGQ GVPINTNSSP DDQIGYYRRA TRRIRGGDGK |
| 101 | MKDLSPRWYF YYLGTGPEAG LPYGANKDGI IWVATEGALN TPKDHIGTRN |
| 151 | PANNAAIVLQ LPQGTTLPKG FYAEGSRGGS QASSRSSSRS RNSSRNSTPG |
| 201 | SSRGTSPARM AGNGGDAALA LLLLDRINQL ESKMSGKGQQ QQGQTVTKKS |
| 251 | AAEASKKPRQ KRTATKAYNV TQAFGRRGPE QTQGNFGDQE LIRQGTDYKH |
| 301 | WPQIAQFAPS ASAFFGMSRI GMEVTPSGTW LTYTGAIKLD DKDPNFKDQV |
| 351 | ILLNKHIDAY KTFPPTEPKK DKKKKADETQ ALPQRQKKQQ TVTLLPAADL |
| 401 | DDFSKQLQQS MSSADSTQA |

The amino acids at positions 203 and 204 are in bold font (and underlined).

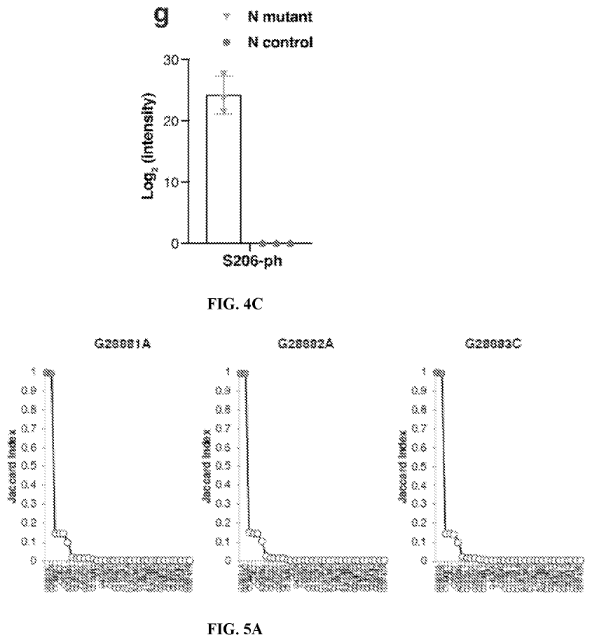

In preferred embodiments, the N protein contains at least one mutation at amino acid position 203 (e.g., R203K) or amino acid position 204 (e.g., G204R). In further preferred embodiments, the N protein contains both mutations R203K and G204R (R203K/G204R N protein mutant). The R203K/G204R mutations in the SARS-CoV-2 N protein are within the linkage region (LKR) containing a serine/arginine-rich motif (SR-rich motif) (FIG. 4A).

Compositions for enriching, isolating, and/or detecting SARS-CoV-2 viruses are provided. The compositions are particularly effective for the rapid and sensitive detection and/or quantitation of SARS-CoV-2 viruses within biological samples, such as sputum samples. The systems and compositions identify SARS-CoV-2 viruses within the sample if they possess viral RNAs.

Therefore, in some embodiments, the nucleocapsid protein has the R203K substitution, as set forth in SEQ ID No. 2:

| | |
|---|---|
| 1 | MSDNGPQNQR NAPRITFGGP SDSTGSNQNG ERSGARSKQR RPQGLPNNTA |
| 51 | SWFTALTQHG KEDLKFPRGQ GVPINTNSSP DDQIGYYRRA TRRIRGGDGK |
| 101 | MKDLSPRWYF YYLGTGPEAG LPYGANKDGI IWVATEGALN TPKDHIGTRN |
| 151 | PANNAAIVLQ LPQGTTLPKG FYAEGSRGGS QASSRSSSRS RNSSRNSTPG |
| 201 | SSKGTSPARM AGNGGDAALA LLLLDRLNQL ESKMSGKGQQ QQGQTVTKKS |
| 251 | AAEASKKPRQ KRTATKAYNV TQAFGRRGPE QTQGNFGDQE LIRQGTDYKH |
| 301 | WPQIAQFAPS ASAFFGMSRI GMEVTPSGTW LTYTGAIKLD DKDPNFKDQV |
| 351 | ILLNKHIDAY KTFPPTEPKK DKKKKADETQ ALPQRQKKQQ TVTLLPAADL |
| 401 | DDFSKQLQQS MSSADSTQA |

The amino acids at positions 203 and 204 are in bold font.

In some embodiments, the nucleocapsid protein has the G204R substitution, as set forth in SEQ ID No. 3:

| | |
|---|---|
| 1 | MSDNGPQNQR NAPRITFGGP SDSTGSNQNG ERSGARSKQR RPQGLPNNTA |
| 51 | SWFTALTQHG KEDLKFPRGQ GVPINTNSSP DDQIGYYRRA TRRIRGGDGK |
| 101 | MKDLSPRWYF YYLGTGPEAG LPYGANKDGI IWVATEGALN TPKDHIGTRN |
| 151 | PANNAAIVLQ LPQGTTLPKG FYAEGSRGGS QASSRSSSRS RNSSRNSTPG |
| 201 | SSRRTSPARM AGNGGDAALA LLLLDRLNQL ESKMSGKGQQ QQGQTVTKKS |
| 251 | AAEASKKPRQ KRTATKAYNV TQAFGRRGPE QTQGNFGDQE LIRQGTDYKH |
| 301 | WPQIAQFAPS ASAFFGMSRI GMEVTPSGTW LTYTGAIKLD DKDPNFKDQV |
| 351 | ILLNKHIDAY KTFPPTEPKK DKKKKADETQ ALPQRQKKQQ TVTLLPAADL |
| 401 | DDFSKQLQQS MSSADSTQA |

The amino acids at positions 203 and 204 are in bold font.

In preferred embodiments, the nucleocapsid protein has both R203K and G204R substitutions, as set forth in SEQ ID No. 4:

| | |
|---|---|
| 1 | MSDNGPQNQR NAPRITFGGP SDSTGSNQNG ERSGARSKQR RPQGLPNNTA |
| 51 | SWFTALTQHG KEDLKFPRGQ GVPINTNSSP DDQIGYYRRA TRRIRGGDGK |
| 101 | MKDLSPRWYF YYLGTGPEAG LPYGANKDGI IWVATEGALN TPKDHIGTRN |
| 151 | PANNAAIVLQ LPQGTTLPKG FYAEGSRGGS QASSRSSSRS RNSSRNSTPG |
| 201 | SSRKTSPARM AGNGGDAALA LLLLDRINQL ESKMSGKGQQ QQGQTVTKKS |
| 251 | AAEASKKPRQ KRTATKAYNV TQAFGRRGPE QTQGNFGDQE LIRQGTDYKH |
| 301 | WPQIAQFAPS ASAFFGMSRI GMEVTPSGTW LTYTGAIKLD DKDPNFKDQV |
| 351 | ILLNKHIDAY KTFPPTEPKK DKKKKADETQ ALPQRQKKQQ TVTLLPAADL |
| 401 | DDFSKQLQQS MSSADSTQA |

The amino acids at positions 203 and 204 are in bold font.

In some embodiments, the nucleocapsid protein is a functional fragment or functional variant of any of SEQ ID NOs.1-4. The terms “functional fragment” and “functional variant” mean any fragment or variant of a nucleoprotein that can be, for example, any number of amino acids sufficient to maintain the biological function of binding to viral RNA.

The data set forth in the examples supports the conclusions that the nucleoprotein of SEQ ID NOs.1-4 specifically and selectively binds to viral RNA from SARS-CoV-2. It may be that the interaction is mediated by interaction of viral RNAs with one or more amino acids within the linkage region (LKR) of the nucleoprotein containing a serine/arginine-rich motif (SR-rich motif). Therefore, in some embodiments, functional fragments and variants retain at least the amino acids within the linkage region (LKR) of the nucleoprotein containing a serine/arginine-rich motif (SR-rich motif). In some embodiments, a fragment is between about 50 and about 419 contiguous amino acids, inclusive of any one of SEQ ID NOs:1-4, or a homologue such as an orthologue or paralogue thereof, or any combination thereof, or any subrange thereof, or any specific integer number of amino acids therebetween, including, but not limited to 50, 60, 75, 100, 150, 200, 250, 300, 350, 400, 410, 415, 416, 417, or 418 contiguous amino acids. Variants can have, for example, at least 65%, 70%, 75%, 80%, 85%, 90%, 95%, 99% or 100% sequence identity to SEQ ID NOs:1, 2, 3, or 4, or a functional fragment thereof, or the corresponding sequence of a homologue such as an orthologue or paralogue of any of the foregoing sequences; or any combination thereof. In a particular embodiment, a variant polypeptide has at least 75%, 80%, 85%, 90%, 95%, 99% or 100% sequence identity to SEQ ID NO:3. In a particular embodiment, a variant polypeptide has at least 75%, 80%, 85%, 90%, 95%, 99% or 100% sequence identity to SEQ ID NO:5. In a particular embodiment, a variant polypeptide has at least 75%, 80%, 85%, 90%, 95%, 99% or 100% sequence identity to SEQ ID NO:7. Preferably variants maintain the ability to interact with viral RNA, i.e., maintain the viral RNA-binding function of nucleoprotein. In some embodiments, a functional nucleoprotein variant is considered to be “functional” if it maintains at least the same specificity and affinity for a specific viral RNA as a “wild-type”, non-modified protein. For example, in some embodiments, nucleoprotein variants are identified as functional if they selectively bind SARS-cov-2 viral RNA and/or co-immuno-precipitate SARS-cov-2 viral RNA to at least equivalent, or approximately equivalent extend to the nucleoprotein of SEQ ID No. 1.

### 2. Capture Tags

Capture proteins include one or more capture tags for selectively isolating the capture protein. In some embodiments, capture proteins include a multiplicity of capture tags. Capture tags are typically covalently bound to the nucleoprotein. Preferably, capture tags do not occlude, diminish, or otherwise impact binding of viral RNA to the nucleoprotein. For example, capture tags are typically bound to nucleoprotein at one or more sites located at a distance from the viral RNA binding site. Preferably, the size and chemical composition of the capture tag facilitate purification of the capture protein in complex with bound viral RNA.

Thus, in some embodiments, the N protein, or a functional fragment or variant thereof, preferably having one or both of R203K and G204R mutations, is covalently conjugated to one or more capture tags. Exemplary capture tags include hexa-histidine tag, FLAG tag, Strep tag, streptavidin-binding peptide (SBP) tag, calmodulin-binding peptide (CBP), glutathione S-transferase (GST), maltose-binding protein (MBP), S-tag, the hemagglutinin (HA) tag, and c-Myc tag. In preferred embodiments, the capture tag is a Strep II tag including eight amino acids (Trp-Ser-His-Pro-Gln-Phe-Glu-Lys).

### 3. Support Matrices

Capture proteins can be immobilized to a support matrix, e.g., a solid phase. For example, the N protein or a functional fragment or variant thereof, preferably having one or both of R203K and G204R mutations, can be covalently or non-covalently immobilized onto a solid phase. Exemplary solid supports include glass plates, microtiter well plates, magnetic beads, silicon wafers and additional solid substrates identifiable by a skilled person upon reading of the present disclosure. Additional exemplary solid supports are particles, preferably magnetic particles. In preferred embodiments, support matrices are magnetic beads.

The surface of the solid support may be hydrophobic or hydrophilic. Preferred are materials presenting a high surface area for binding of the nucleocapsid proteins, and subsequently of the nucleic acid. Such supports will generally have an irregular surface and may be for example be porous or particulate e.g., particles, fibers, webs, sinters, or sieves. Particulate materials e.g., beads are generally preferred due to their greater binding capacity, particularly polymeric beads/particles.

In some embodiments, support matrices are spherical beads. The size of the beads is not critical, but they may for example be of the order of diameter of at least 1 μm and preferably at least 2 μm and have a maximum diameter of preferably not more than 10 μm and more preferably not more than 6 μm, for example, beads of diameter 2.5 μm, 3.0 μm, 3.5 μm, 4.0 μm, and 4.5 μm. Monodisperse particles, that is those which are substantially uniform in size (e.g., size having a diameter standard deviation of less than 50) have the advantage that they provide very uniform reproducibility.

In some embodiments, the capture proteins are immobilized to resins or matrices on an affinity chromatography. Exemplary resins or matrices are agarose or magnetic agarose beads. Capture proteins can be immobilized to resins or matrices on an affinity chromatography via their capture tags. For example, the capture protein having a Strep-TAG® II can be immobilized to affinity chromatography via interactions with resins or matrices associated or conjugated to streptavidin, e.g., Strep-TACTIN®.

### B. Samples

The compositions selectively enrich SARS-CoV-2 virus RNA present within a test or a sample. In some embodiments, a sample is diluted, concentrated, or otherwise obtained from a liquid, gel, emulsion, or a solid, such as a powder. In some embodiments, samples are biological samples, such as those obtained from the body of subject. In other embodiments, samples are environmental samples, such as those obtained from a water source. Samples are typically fluids, such as biological fluids. In some embodiments, samples are frozen and/or lyophilized.

Samples for use in the described assays can originate from any source, including liquids, frozen liquids, or powders, such as freeze-dried or lyophilized samples. In some embodiments, the sample is obtained from a biological source, such as one or more tissues, cells, or bodily fluids of a subject. In other embodiments, the sample is obtained from an environmental source, such as a sample of water, ice, soil, or a sample obtained from a non-biological source, such as the surface of an object. In some embodiments, the sample is obtained from a biological fluid of undetermined origin from a subject. In some embodiments, the origin of the sample is a human patient, such as a patient identified or suspected of having a disease, such as a respiratory or circulatory disease or disorder. Therefore, in preferred embodiments, a sample for use in the described assays for detection and/or quantitation of SARS-CoV-2 virus is obtained by enrichment and isolation of RNA from a biological sample of bodily fluids taken from a human subject. Exemplary bodily fluids include sputum, saliva, mucus, blood, serum, tears, sweat, urine, semen, fluids from the respiratory tract, gastric fluid, fluids from the digestive tract, fluids from the urogenital tract, spinal fluid, ocular fluid, synovial fluid, feces, pus, bile, or other biological fluid. In some embodiments, a sample contains a mixture of two or more biological fluids. In some embodiments, a biological sample from a patient contains biological fluids of undetermined origin from a subject. In some embodiments, a sample contains biological fluids of undetermined origin from a subject.

In some embodiments, the sample is contained within in a container, or together with one or more devices used to obtain the sample, such as a swab, syringe, and cotton bud, inoculating loop or other apparatus for obtaining a biological sample from a subject. Therefore, in some embodiments, the sample includes one or more components associated with the collection device.

In some embodiments, the sample includes a diluent, filler, excipient, or preservative. In some embodiments, the sample includes one or more reagents which function to preserve or maintain the SARS-CoV-2 virus within the sample. In some embodiments, the sample includes one or more reagents that prevent or reduce the activity of RNase enzymes.

### 1. Viral RNAs

In some embodiments, samples include viral RNAs. Compositions of capture proteins enrich, isolate, and/or detect viral RNAs from a sample. In some embodiments, the compositions selectively identify RNAs derived from specific viruses, particularly those from the SARS-CoV-2 viruses, which are coronaviruses of the subgenus Sarbecovirus. Compositions of capture proteins conjugated or complexed with viral RNAs are provided. The viral RNAs are specifically and selectively bound to the capture proteins. Complexes including viral RNAs specifically bound to capture proteins are provided.

a. Coronaviruses

The coronaviruses (order Nidovirales, family Coronaviridae, and genus Coronavirus) are a diverse group of large, enveloped, positive-stranded RNA viruses that cause respiratory and enteric diseases in humans and other animals (Rota, et al., Science, May 2003, Page 1/10.1126/1085952).

Coronaviruses typically have narrow host and can cause severe disease in many animals, and several viruses, including infectious bronchitis virus, feline infectious peritonitis virus, and transmissible gastroenteritis virus, are significant veterinary pathogens. Human coronaviruses (HCoVs) are found in both group 1 (HCoV-229E) and group 2 (HCoV-OC43) and are historically responsible for ˜30% of mild upper respiratory tract illnesses.

At ˜30,000 nucleotides, their genome is the largest found in any of the RNA viruses. There are three groups of coronaviruses; groups 1 and 2 contain mammalian viruses, while group 3 contains only avian viruses. Within each group, coronaviruses are classified into distinct species by host range, antigenic relationships, and genomic organization. The genomic organization is typical of coronaviruses, with the characteristic gene order (5′-replicase [rep], spike [S], envelope [E], membrane [M], nucleocapsid [N]-3′) and short untranslated regions at both termini. The SARS-CoV rep gene, which comprises approximately two-thirds of the genome, encodes two polyproteins (encoded by ORF1a and ORF1b) that undergo co-translational proteolytic processing. There are four open reading frames (ORFs) downstream of rep that are predicted to encode the structural proteins, S, E, M, and N, which are common to all known coronaviruses.

i. SARS-CoV-2

The systems and compositions identify the SARS-CoV-2 betacoronavirus of the subgenus Sarbecovirus. SARS-CoV-2 viruses share approximately 79% genome sequence identity with the SARS-CoV virus identified in 2003. The genome organization of SARS-CoV-2 viruses is shared with other betacoronaviruses; six functional open reading frames (ORFs) are arranged in order from 5′ to 3′: replicase (ORF1a/ORF1b), spike (S), envelope (E), membrane (M) and nucleocapsid (N).

ii. Capture Protein/SARS-CoV-2 RNA Complex

The systems and compositions selectively enrich SARS-CoV-2 virus RNA present within a test or a sample, preferably by forming a complex with the capture protein. Thus, in some embodiments, compositions of a capture protein/SARS-CoV-2 RNA complex are also described.

### III. Methods of Use

### A. Methods for Enriching and Isolating Viral RNAs

The methods of using nucleocapsid is the nucleocapsid (N) protein derived from SARS-CoV-2 virus, or a functional fragment or variant thereof, preferably having one or both of R203K and G204R mutations, are also described. In some embodiments, the methods enrich RNA molecules from a sample, preferably viral RNAs. In one embodiment, the viral RNA to be enriched is RNAs from SARS Covid-2.

Methods of enrichment and isolation of viral RNA are described. A typical method for enriching viral RNAs includes one or more of the steps of
i) contacting a sample of interest with a capture protein, preferably immobilized to a solid phrase; optionally
ii) removing supernatant; and further optionally
iii) washing to remove unbound and/or non-specific binding (this leaves a sample enriched in RNA bound to mutant form of N-protein); and
iv) isolating the capture protein complexed with viral RNA, optionally
v) eluting isolated viral RNA from the capture protein using an appropriate buffer; and optionally
vi) Quantitating the isolated viral RNA.

All of the described steps can be carried out using N proteins derived from SARS-CoV-2 virus, preferably having R203K and G204R mutations (mutant R203K/G204R N protein), that are immobilized on a solid phase. For example, the mutant R203K/G204R N protein can be covalently or non-covalently immobilized onto a solid phase.

The methods are advantageously amenable to automation, particularly if particles, and especially, magnetic particles are used as the solid support.

### 1. Sample Binding

To aid manipulation and separation, magnetic beads are preferred. The term “magnetic” as used herein means that the support is capable of having a magnetic moment imparted to it when placed in a magnetic field, and thus is displaceable under the action of that field. Thus, a support matrix including magnetic particles may readily be removed by magnetic aggregation, which provides a quick, simple, and efficient way of separating the particles following the binding steps, and is a far less rigorous method than traditional techniques such as centrifugation which generate shear forces which may disrupt nucleic acids. Thus, the magnetic particles after sample binding may be removed onto a suitable surface by application of a magnetic field e.g., using a permanent magnet. It is usually sufficient to apply a magnet to the side of the vessel containing the sample mixture to aggregate the particles to the wall of the vessel and to remove the remainder of the sample for further steps.

In some embodiments, the methods are carried out within the wells of a microtiter plate.

In preferred embodiments, buffers and reagents suitable for binding will provide a final pH of 7.5 in the mixture. In one embodiment, a suitable buffer for binding includes 0.15 M to 0.5 M NaCl, 10 to 50 mM Tris-HCl (pH 7.5), 1 to 5 mM EDTA, and RNase inhibitors. In a further embodiment, a suitable buffer for binding includes 50 mM Tris HCl pH7.5, 150 mM KCl, 0.1 mM EDTA, 1 mM DTT, 5% Glycerol, 0.02% NP40, 1 mM PMSF, and RNase inhibitors.

### 2. Washing

When RNAs are subsequently bound to a solid phase, the methods can include one or more steps of washing the solid phase to remove unbound molecules and/or non-specific binding molecules.

Suitable washing buffers retain the binding of viral RNA to the N protein and wash away unbound or non-specifically bound molecules. In one embodiment, a suitable buffer for binding includes 0.15 M to 0.5 M NaCl, 10 to 50 mM Tris-HCl (pH 7.5), 1 to 5 mM EDTA, and RNase inhibitors. In a further embodiment, a suitable buffer for binding includes 50 mM Tris HCl pH7.5, 150 mM KCl, 0.1 mM EDTA, 1 mM DTT, 5% Glycerol, 0.02% NP40, 1 mM PMSF, and RNase inhibitors.

### 3. Elution

RNA samples can be released from the capture surface of the solid phase (e.g., magnetic beads) using a suitable buffer. Preferably, RNA is eluted from the solid surface in a manner than minimizes loss and/or manipulation of the RNA. Exemplary methods and suitable buffers for eluting RNA bound to solid phase matrices are known in art. An exemplary elution is using Trizol as described in the Examples.

### B. Methods for Detecting and Quantifying Viral RNAs

Methods for detecting and quantifying viral RNA, particularly SARS-CoV-2 RNA in biological samples are also provided. In some embodiments, the methods employ RT-qPCR or RNA-Seq using the viral RNAs enriched and isolated using the described methods.

In some embodiments, the detecting step includes steps for quantifying and/or recording the number of copies of viral target RNAs within the sample. Typically, the sample may contain 10 copies, 100 copies, 1,000 copies, 2,000 copies, 3,000 copies, 4,000 copies, 5,000 copies, 6,000 copies, 7,000 copies, 8,000 copies, 8,000 copies, 10,000 copies, or more than 10,000 copies of viral genomic RNAs.

### 1. Quantitative PCR

Methods for detecting and quantifying SARS-CoV-2 nucleic acid from within biological samples using a RT-qPCR system are described. Methods can detect the presence of SARS-CoV-2 within an input sample, typically isolated RNA extracted from the sample using methods described above. The methods include contacting the isolated RNA extracted from the sample with a reaction mixture also which includes
(i) a set of primers configured to amplify one or more fragments of the SARS-CoV-2 virus, preferably against viral N gene (N1 and N2), E gene, S gene, and/or ORF1ab region;
(ii) a probe configured to bind to the amplified nucleic acid fragment; and
(iii) a RT-qPCR reaction mixture including reagents necessary for amplifying the one or more fragments of the SARS-CoV-2 virus.

The methods incubate the reaction mixture under conditions sufficient for an RT-qPCR reaction to amplify the one or more fragments of the SARS-CoV-2 virus to create an output sample.

The methods detect the one or more fragments of the SARS-CoV-2 virus and probe within the output sample,
wherein the presence of the one or more fragments of the SARS-CoV-2 virus and probe within the output sample identifies the input sample as containing SARS-CoV-2.

Typically, the contacting step occurs within a thermal cycler or other apparatus suitable for conducting and monitoring necessary for conducting an RT-qPCR procedure.

In some embodiments, the detecting step includes steps for quantifying and/or recording the number of copies of viral target RNAs within the sample.

In preferred embodiments, the composition for RNA-qPCR includes (i) nucleic acid oligonucleotide primers and (ii) a nucleic acid probe specific against viral N gene (N1 and N2), E gene, S gene, and/or ORF1ab region (Table 2).

### 2. Diagnosis of COVID-19

The described methods are useful for detecting the presence of the SARS-CoV-2 viruses within a sample obtained from a subject, such as a patient who is identified as having, or is suspected as having COVID-19. Therefore, in some embodiments, the methods diagnose a subject as having an infection with SARS-CoV-2 viruses, and/or having COVID-19.

In some embodiments, a subject is selected if they are suspected of being, or are identified as at risk of being infected with a coronavirus virus, for example, a SARS-CoV-2 virus. In some embodiments, subjects are selected based on one or more symptoms or other indications in the subject. In a preferred embodiment, a subject has one or more symptoms or physiological markers of COVID-19. Symptoms of COVID-19 include but are not limited to fever, fatigue, dry cough, sputum production, headache, haemoptysis, diarrhoea, anorexia, sore throat, chest pain, chills, nausea, vomiting, dyspnoea, pneumonia, respiratory failure, septic shock, multiple organ dysfunction or failure and olfactory and taste disorders, such as loss or alteration of smell and/or taste. In some embodiments, subjects are asymptomatic. In some embodiments, asymptomatic subjects are selected without additional indications, for example, as a part of a community or population-wide screening process. In other embodiments, asymptomatic subjects are selected due to an increased risk of developing COVID-19, for example, due to potential exposure to SARS-CoV-2 virus or to proximity to infected individuals. In some embodiments, the same subject is repeatedly screened for the presence of SARS-CoV-2 viruses, for example, every day, week, month, or year.

In all of the described methods, the methods can include one or more steps of identifying a subject for screening according to the described methods for detecting the presence of the SARS-CoV-2 viruses within a sample obtained from the subject. Therefore, in some embodiments, the methods include a step for selecting a subject in need of screening for infection with a coronavirus. In further embodiments, the methods include one or more steps of obtaining one or more biological samples from the subject.

### C. Methods of Treatment

In some embodiments, the methods further include a step of treating subjects identified as having an active infection of SARS-CoV-2.

In some embodiments, the methods administer to the subject identified as suitable for receiving antiviral and/or monoclonal therapies an effective amount of antiviral and/or monoclonal therapies to treat or prevent one or more symptoms of coronavirus infection in the subject, for example, reducing or preventing one or more symptoms or physiological markers of severe acquired respiratory syndrome (SARS) in a subject. Exemplary symptoms of COVID-19 include cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath. Therefore, in some embodiments, the methods prevent or reduce one or more of cough, fatigue, fever, body aches, headache, sore throat, loss or altered sense of taste and/or smell, vomiting, diarrhea, cytokine storm, skin changes, ocular complications, confusion, chronic neurological impairment, chest pain and shortness of breath.

### 1. Therapeutic Agents

Remdesivir (GS-5734), an inhibitor of the viral RNA-dependent, RNA polymerase with in vitro inhibitory activity against SARS-CoV-1 and the Middle East respiratory syndrome (MERS-CoV), was identified early as a promising therapeutic candidate for Covid-19 because of its ability to inhibit SARS-CoV-2 in vitro. On Oct. 22, 2020, the U.S. Food and Drug Administration (FDA) approved the antiviral drug VEKLURY® (remdesivir) for use in adults and pediatric patients (12 years of age and older and weighing at least 40 kg) for the treatment of COVID-19 requiring hospitalization.

The FDA has issued an Emergency Use Authorization (EUA) on Dec. 22, 2021 for Pfizer's PAXLOVID™ (nirmatrelvir tablets and ritonavir tablets, co-packaged for oral use) for the treatment of mild-to-moderate coronavirus disease 2019 (COVID-19) in adults and pediatric patients (12 years of age and older weighing at least 40 kg) with positive results of direct severe acute respiratory syndrome coronavirus 2 (SARS-CoV-2) viral testing, and who are at high risk for progression to severe COVID-19, including hospitalization or death. PAXLOVID™ consists of nirmatrelvir, which inhibits a SARS-CoV-2 protein to stop the virus from replicating, and ritonavir, which slows down nirmatrelvir's breakdown to help it remain in the body for a longer period at higher concentrations. PAXLOVID™ is administered as three tablets (two tablets of nirmatrelvir and one tablet of ritonavir) taken together orally twice daily for five days, for a total of 30 tablets. PAXLOVID™ is not authorized for use for longer than five consecutive days.

Monoclonal antibody therapies remain available under EUA, including REGEN-COV® (casirivimab and imdevimab, administered together), and bamlanivimab and etesevimab, administered together.

FDA has authorized the emergency use of baricitinib to treat COVID-19 in hospitalized adults and pediatric patients 2 years or older requiring supplemental oxygen, non-invasive or invasive mechanical ventilation, or extracorporeal membrane oxygenation (ECMO). According to a statement issued by the WHO on January 14, Baricitinib is recommended for treating patients suffering with severe or critical Covid-19. On the other hand, Sotrovimab, a monoclonal antibody drug, is recommended for treating patients who have mild or moderate Covid-19.

Accordingly, in some embodiments, the methods involve the step of administering one or more of antiviral drugs such as remdesivir and PAXLOVID™, and monoclonal antibodies such as casirivimab, imdevimab, bamlanivimab, etesevimab, baricitinib, and sotrovimab, to a subject identified as having an elevated risk of developing one or more symptoms associated with severe COVID-19 based on the disclosed methods. The methods administer an effective amount of antiviral drugs and/or monoclonal antibodies to prevent, retard the development of, and/or treat one or more symptoms associated with coronavirus infection in the subject, for example, reducing or preventing one or more symptoms or physiological markers of severe acquired respiratory syndrome (SARS) in a subject.

### 2. Additional Therapies and Procedures

The antiviral drugs and/or monoclonal antibodies against SARS-CoV-2 can be administered alone or in combination with one or more additional therapies. In some embodiments, the combination therapy includes administration of one or more of the antiviral drugs and/or monoclonal antibodies in combination with one or more additional active agents. The combination therapies can include administration of the active agents together in the same admixture, or in separate admixtures. Therefore, in some embodiments, the pharmaceutical composition includes two, three, or more active agents. Such formulations typically include an effective amount of an agent targeting the site of treatment. The additional active agent(s) can have the same or different mechanisms of action. In some embodiments, the combination results in an additive effect on the treatment of the lung condition. In some embodiments, the combinations result in a more than additive effect on the treatment of the disease or disorder.

The additional therapy or procedure can be simultaneous or sequential with the administration of the dendrimer composition. In some embodiments, the additional therapy is performed between drug cycles or during a drug holiday that is part of the dosage regime. For example, in some embodiments, the additional therapy or procedure is damage control surgery, fluid resuscitation, blood transfusion, bronchoscopy, and/or drainage.

In some embodiments, the antiviral drugs and/or monoclonal antibodies are used in combination with oxygen therapy. In further embodiments, the additional therapy or procedure is prone positioning, recruitment maneuver, inhalation of NO, extracorporeal membrane oxygenation (ECMO), intubation, and/or inhalation of PGI2. A prone position enhances lung recruitment in a potentially recruitable lung by various mechanisms, releasing the diaphragm, decreasing the effect of heart and lung weight and shape on lung tissue, decreasing the lung compression by the abdomen, and releasing the lower lobes, which improves gas exchange and decreases mortality in severe ARDS patients. ECMO provides extracorporeal gas exchange with no effect on lung recruitment. It affords lung rest and works well for the non-recruitable lung. It has been shown to improve survival for certain groups of patients in high-performance ECMO centers.

In some embodiments, the compositions and methods are used prior to or in conjunction, subsequent to, or in alternation with treatment with one or more additional therapies or procedures.

One or more additional therapeutic, diagnostic, and/or prophylactic agents may be used to treat inflammation in the lungs, and/or systemic inflammation resulting from COVID-19 induced pneumonia. Additional therapeutic agents can also include one or more of antibiotics, surfactant, corticosteroids, and glucocorticoids.

In some embodiments, the composition may contain one or more additional compounds to relief symptoms such as inflammation, or shortness of breath.

In some embodiments, one or more agents include bronchodilators, corticosteroids, methylxanthines, phosphodiesterase-4 inhibitors, anti-angiogenesis agents, antibiotics, antioxidants, anti-viral agents, anti-fungal agents, anti-inflammatory agents, immunosuppressant agents, and/or anti-allergic agents, are administered prior to, in conjunction with, subsequent to, or alternation with treatment with the disclosed antiviral drugs and/or monoclonal antibodies.

The amount of a second therapeutic generally depends on the severity of lung disorders to be treated. Specific dosages can be readily determined by those of skill in the art. See Ansel, Howard C. et al. Pharmaceutical Dosage Forms and Drug Delivery Systems (6^{th} ed.) Williams and Wilkins, Malvern, PA (1995).

The additive drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

In some embodiments, the additional agent is a diagnostic agent imaging or otherwise assessing the site of application. Exemplary diagnostic agents include paramagnetic molecules, fluorescent compounds, magnetic molecules, and radionuclides, x-ray imaging agents, and contrast media. These may also be ligands or antibodies which are labelled with the foregoing or bind to labelled ligands or antibodies which are detectable by methods known to those skilled in the art.

In certain embodiments, the pharmaceutical composition contains one or more local anesthetics. Representative local anesthetics include tetracaine, lidocaine, amethocaine, proparacaine, lignocaine, and bupivacaine. In some embodiments, one or more additional agents, such as a hyaluronidase enzyme, is also added to the formulation to accelerate and improves dispersal of the local anesthetic.

In some embodiments, the methods include steps of monitoring progress of treatment, and/or to detect SARS-CoV-2 viral RNA following the treatment. In further embodiments, the method further includes discontinuing treatment of the subject if the quantity of SARS-CoV-2 viral RNA is reduced, for example by 10%, 20%, 30%, 40%, 50%, 60%, 70%, 80%, 90%, or more than 90% compared to the level prior to treatment.

The methods can be used to test for presence of the SARS-CoV-2 viral RNA over a period of time, or after the initial negative or positive read-out, for example, over a week, two weeks, three weeks, four weeks, a month, two months, three months, four months, five months, six months, about a year, two years, three years, four years, five years, more than five years.

### D. Controls

In some embodiments, the method includes one or more control samples which act as a control for the specific detection and/or quantification of the SARS-CoV-2 virus within. Exemplary negative control samples include purified RNA or DNA derived from viruses that share little or no genetic relatedness with the SARS-CoV-2 virus. Exemplary negative control viruses include RNA extracted from human coronaviruses 229E, OC43, HKU1, NL63, and OC43, MERS, camel coronavirus HKU23, human influenza A viruses (H1N1, H3N2, H5N1, and H7N9 subtypes), avian influenza (H1, H4, H6, and H9 subtypes), human influenza B viruses (Yamagata and Victoria lineages), and adenovirus, enterovirus, human parainfluenza viruses (PIV1, 2, 3 and 4), respiratory syncytial virus, human metapneumovirus, rhinovirus and human bocavirus. In some embodiments, negative controls can include RNA extracted from retrospective human respiratory specimens previously tested positive for any of these viruses. In some embodiments, the negative controls are recombinantly-produced nucleic acid vectors which lack one or more of the nucleic acid sequences required for the activity of the designed primer and probe sets that are to be used. In other embodiments, RNA extracted from sputum samples from patients without respiratory viral infections are negative controls.

In some embodiments, positive controls to confirm the specificity and efficacy of the assay for detecting and quantifying the SARS-CoV-2 virus include viral RNA extracted from SARS-CoV-2-infected cells, as well as the RT-PCR products of SARS coronavirus generated by the viral N gene (N1 and N2), E gene, S gene, and/or ORF1ab gene, cloned into plasmids.

In some embodiments, the control samples are serially diluted, to evaluate the performance of the assays.

### IV. Kits

The materials described above as well as other materials can be packaged together in any suitable combination as a kit useful for performing, or aiding in the performance of, the disclosed method. It is useful if the kit components in each kit are designed and adapted for use together in the disclosed method. Kits for including reagents necessary to conduct the methods are also provided.

Typically, the kits include the nucleocapsid (N) protein derived from SARS-CoV-2 virus, or a functional fragment or variant thereof, preferably having one or both of R203K and G204R mutations, immobilized to a solid phrase such as magnetic beads and/or affinity-binding column, wash buffer, elution buffer, and instructions for carrying out the methods. In some embodiments, the kit also includes a positive control sample and/or a negative control sample.

The present invention will be further understood by reference to the following non-limiting examples.

### EXAMPLES

### Example 1: SARS-CoV-2 Genomes from Saudi Arabia Implicate Nucleocapsid Mutations in Host Response and Increased Viral Load

### Methods

Sample collection. As part of the study, nasopharyngeal swab samples were collected in 1 ml of TRIzol (Ambion, USA) from 892 COVID-19 patients with various grades of clinical disease manifestations-consisting of severe, mild, and asymptomatic symptoms. The anonymized samples were amassed from 8 hospitals and one quarantine hotel located in Madinah, Makkah, Jeddah, and Riyadh. Patient metadata in the form of age, sex, comorbidities, ICU submission, and mortality were provided by the hospitals and used for statistical analysis. Ethical approvals were obtained from the Institutional review board of the Ministry of Health in the Makkah region with the numbers H-02-K-076-0420-285 and H-02-K-076-0320-279, as well as the Institutional review board of Dr. Sulaiman Al Habib Hospital number RC20.06.88 for samples from Riyadh and the Eastern regions respectively.

RNA Isolation. RNA was extracted using the Direct-Zol RNA Miniprep kit (Zymo Research, USA) following the manufacturer's instructions, along with several optimization steps to improve the quality and quantity of RNA from clinical samples. The optimization included extending the TRIzol incubation period, and the addition of chloroform during initial lysis step to obtain the aqueous RNA layer. The quality control of purified RNA was performed using Broad Range Qubit kit (Thermo Fisher, USA) and RNA 6000 Nano LabChip kit (Agilent, USA) respectively. RT-PCR was conducted using the one-step Super Script III with Platinum Taq DNA Polymerase (Thermo Fisher, USA) and TaqPath COVID-19 kit (Applied Biosystems, USA) on the QuantStudio 3 Real-Time PCR instrument (Applied Biosystems, USA) and 7900 HT ABI machine. The primers and probes used were targeting two regions in the nucleocapsid gene (N1 and N2) in the viral genome following the Centre for Disease Control and prevention diagnostic panel, along with primers and probe for human RNase P gene (CDC; fda.gov/media/134922/download) (Table 1 and Table 2). Samples were considered COVID positive once the cycle threshold (Ct) values for both N1 and N2 regions were less than 40. For amplicon seq purposes, the samples chosen were of Ct less than 35 to ensure successful genome assembly in order to upload on GISAID.

TABLE 1
Primers used for cloning

| Primer Name | Sequence 5′ to 3′ |
|---|---|
| pLVX-N1-F1 | CTATTTCCGGTGAATTCGCCG |
| | (SEQ ID No. 5) |
| pLVX-N1-R1 | GGGGGGGGATCCTTACTTTTC |
| | (SEQ ID No. 6) |
| pLVX-N1-Mut-F1 | CCAGGGTCCAGTAAACGAACAAGTCCGGCGC |
| | (SEQ ID No. 7) |
| pLVX-N1-Mut-R1 | GCGCCGGACTTGTTCGTTTACTGGACCCTGG |
| | (SEQ ID No. 8) |

TABLE 2
Primers and Probe for SARS-CoV-2

| Name | Catalog Number |
|---|---|
| nCOV_N1 Forward Primer | 10006821 |
| Aliquot, 50 nmol | |
| nCOV_N1 Reverse Primer | 10006822 |
| Aliquot, 50 nmol | |
| nCOV_N1 Probe Aliquot, | 10006823 |
| 25 nmol | |
| nCOV_N2 Forward Primer | 10006824 |
| Aliquot, 50 nmol | |
| nCOV_N2 Reverse Primer | 10006825 |
| Aliquot, 50 nmol | |
| nCOV_N2 Probe Aliquot, | 10006826 |
| 25 nmol | |
| E gene E_Sarbeco_F | ACAGGTACGTTAATAGTTAATAGCGT |
| | (SEQ ID No.9) |
| E_Sarbeco_R | ATATTGCAGCAGTACGCACACA |
| | (SEQ ID No. 10) |
| E_Sarbeco_Pl | FAM-ACACTAGCCATCCTTACTGCGCTTCG-BBQ |
| | (SEQ ID No. 11) |
| 2019-nCoV (ORF1ab) (FAM dye) | TaqMan ™ 2019-nCoV Assay Kit v1 |
| | (Cat. No. A47532) |
| 2019-nCoV (S Gene) (FAM dye) | TaqMan ™ 2019-nCoV Assay Kit v1 |
| | (Cat. No. A47532) |

Sequencing and data analysis. cDNA and amplicon libraries were prepared using the COVID-19 ARTIC-V3 protocol, producing ˜ 400 bp amplicons tiling the viral genome using V3 nCoV-2019 primers (Wellcome Sanger Institute, UK; dx.doi.org/i0.17504/protocols.io.beuzjex6). Amplicons were then processed for deep, paired-end sequencing with the Novaseq 6000 platform on the SP 2×250 bp flow cell type (Illumina, USA).

Genome assembly, SNP and indel calling. Illumina adapters and low-quality sequences were trimmed using Trimmomatic (v0.38)⁶³. Reads were mapped to SARS-CoV-2 Wuhan-Hu-1 NCBI reference sequence NC_045512.2 using BWA (v0.7.17)⁶⁴. Mapped reads were processed using GATK (v4.1.7) pipeline com-mands MarkDuplicatesSpark, HaplotypeCaller, VariantFiltration, SelectVariants, BaseRecalibrator, ApplyBQSR, and HaplotypeCaller to identify variants⁶⁵ High quality SNPs were filtered using the filter expression: “QD<2.0∥FS>60.0∥SOR>3.0∥MQRankSum<−12.5∥ReadPosRankSum<−8.0” High quality Indels were filtered using the filter expression: “QD<2.0∥FS>200.0∥SOR>10.0∥ReadPosRankSum<−20.0” “QD<2.0∥FS>60.0∥SOR>3.0∥MQRankSum<−12.5∥ReadPosRankSum<−8.0” High quality Indels were filtered using the filter expression: “QD<2.0∥FS>200.0∥SOR>10.0∥ReadPosRankSum<−20.0” Consensus sequences were generated by applying the good quality variants from GATK on the reference sequence using bcftools (v1.9) consensus command⁶⁶. Regions which are covered by less than 30 reads are masked in the final assembly with ‘N’s.

Consensus assembly sequences were deposited to GISAID¹¹. To retrieve high-confidence SNPs assembled sequences were re-aligned against the Wuhan-Hu-1 reference sequence (NC_045512.2), and only positions in the sample sequences with unambiguous bases in a 7-nucleotide window centered around the SNP position were kept for further analysis.

Phylogenetic analysis. To generate the phylogeny of Saudi samples with a global context, a total of 308,012 global sequences were downloaded from GISAID on 31 Dec. 2020, filtered and processed using Nextstrain pipeline¹². Global sequences were grouped by country and sample collection month and 20 sequences per group were randomly sampled which resulted in 10,873 global representative sequences and 952 Saudi sequences. The phylogeny was constructed using IQ-TREE (v2.0.5)⁶⁷, clades were assigned using Nextclade and internal node dates were inferred, and sequences pruned using TreeTime (v0.7.5)⁶⁸. Nextstrain protocol was followed for the above-mentioned steps. The resulting global phylogenetic tree was reduced to retain the branches that lead to Saudi leaf nodes and visualized using baltic library (https://github.com/evogytis/baltic).

Phylodynamic analysis. Phylodynamic analyses use the same sequence subset used in the full phylogenetic analysis, extracted from the GISAID SARSCoV-2 database11. Wrapper functions for the importation date estimates and skygrowth model are provided in the sarscov2 R package as ‘compute timports’ and ‘sky-growth1’ respectively (https://github.com/JorgensenD/sarscov2Rutils)⁶⁹

Importation date estimates for Nextstrain clades. Importation rate estimates were carried out using all available sequence data for Saudi Arabia deposited on GISAID¹¹ up to 31 Dec. 2020, including the sequences described in this paper. Sequences were grouped by Nextstrain clade for analysis using the Next-strain_clade parameter¹² in the GISAID metadata table. Additional international sequences were selected for each of the included Nextstrain clade based on Tamura Nei⁹³ distance with the C program tn93 (v1.0.6) (github.com/veg/tn93)⁷⁰. Five hundred sequences were selected from available closely related sequences in a time stratified manner, taking every N/500th sequence from the set of N sequences arranged by date, rounded to the nearest integer.

For each Nextstrain clade a maximum-likelihood phylogeny was produced with IQTree (v1.6.12) with an HKY substitution model^{67,71}. These trees were dated using the R package treedater (v0.5.0) after collapsing short branch lengths and resolving polytomies randomly fifteen times for each clade with the functions di2multi and multi2di from the ape R package (v5.5)^{72,73}. A strict molecular clock was used when estimating dated phylogenies, constrained between 0.0009 and 0.0015 substitutions per site per year74. The state of each internal node in the phylogeny was reconstructed by maximum parsimony with the R package phangorn (v2.7.0)⁷⁵. As this method cannot directly estimate the timing of an importation event, importations were estimated to occur at the midpoint of a branch along which a state change occurs between the internal and external samples.

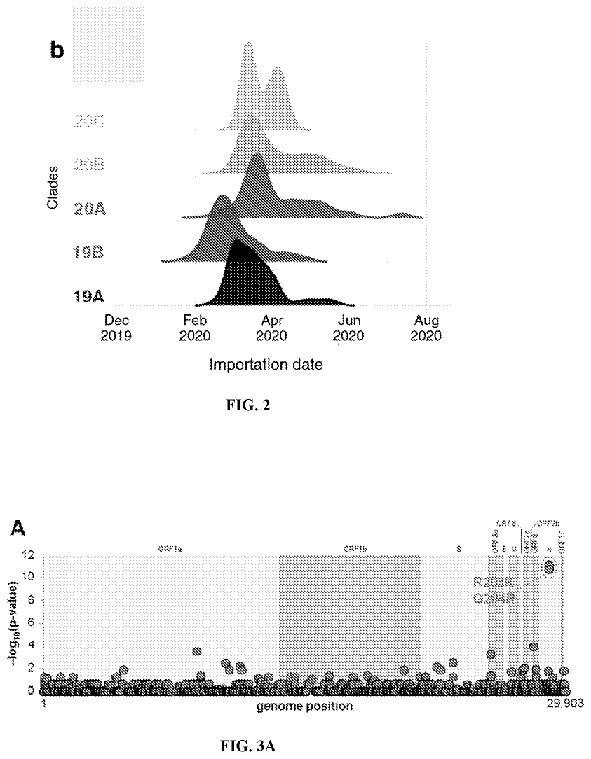

The probability density of importation events over time into Saudi Arabia by cluster is presented in FIG. 2B where a gaussian kernel density estimator is used with a bandwidth of 5.51 chosen using the Silverman rule of thumb, implemented in the geom_density function of the ggplot2 R package (v3.3.5)^{76,77}.

Skygrowth model. The assembled sequences, together with all other reported sequences for Saudi Arabia available on GISAID on 31 Dec. 2020, were used to construct estimates of the effective population size and growth rate of SARS-CoV2 in Saudi Arabia over the course of the first wave of the epidemic (March to September 2020)¹¹. The collected sequences were used to produce a maximum likelihood phylogeny with an HKY substitution model in IQTree (v1.6.12)^{67,71}. A set of 1000 bootstrap pseudo-replicate trees were produced with the ultrafast bootstrap approximation 8. For each bootstrap phylogeny, branches with length less than 10⁻⁵ were collapsed and polytomies were resolved randomly using the di2multi and multi2di functions in the ape R package (v5.5)⁷². These dichotomous trees were produced for subsequent molecular clock analysis and coalescent ana-lysis. The set of 1000 initial bootstrap trees and 1000 additional phylogenies with randomly resolved short branches were dated using the reported collection dates for the sequenced samples as the date of the corresponding tip in each phylogeny. A strict molecular clock was used when estimating dated phylogenies, constrained between 0.0009 and 0.0015 substitutions per site per year with the R package treedater (v0.5.0)^{73,74}

The R package Skygrowth (v0.3.1) was used with these phylogenies to estimate the growth rate and effective population size of SARS-CoV2 in Saudi Arabia over time¹⁶. Skygrowth is a Bayesian non-parametric model of effective population size, with the primary difference to other skyline smoothing methods being that the first-order stochastic process is defined in terms of the growth rate of the effective population size (Ne) rather than Ne itself. The growth rate of Ne often has a simple relationship with the growth rate of the epidemic from which samples are collected.

The model included 35 timesteps and an exponential prior on the smoothing parameter tau (precision) corresponding to a 1% change in growth per week. The growth rate output was converted to an estimate of R over time using an assumed generation time ψ of 9.5 days⁷⁹.

Although phylogenetic methods are sensitive to sampling rate changes over time, the coalescent method used in Skygrowth is relatively robust to heterogenous sampling through time, but can still be biased by unequal sampling in space or risk groups. Computation of reproduction numbers is premised on equivalence of the growth rate of the epidemic and the growth rate ofthe effective population size, which does not hold when the transmission rate is highly variable.

Origin of R203K/G204R SNPs. A total of 590 K samples submitted to GISAID until February 24 were downloaded and SNPs identified by mapping against the Wuhan-Hu-1 reference sequence (NC_045512.2) using minimap2 (v2.17)⁸⁰. The variants were queried to count the distribution of triplets among various Nextstrain clades (Table 3). To identify if there are lineages of triplet SNPs in clades other than 20B, a phylogenetic tree was constructed by including all R203K/G204R samples found in other clades outside 20B and its subclades (Table 4). As it was already evident that 20B and its subclades contains lineages of R203K/G204R samples, subsamples from 20B and its subclades were sufficient to obtain a total of 16,386 samples.

TABLE 3
Counts of partial and full R203K/G204R SNPs at genome position
28,881-28,883, where the Wuhan reference has the GGG genotype.

| SNP | Count |
|---|---|
| GGC | 23 |
| GAG | 47 |
| GAA | 29 |
| AGG | 563 |
| AGC | 27 |
| AAG | 26 |
| AAC | 232,475 |

TABLE 4
Counts of R203K/G204R SNPs in different Nextstrain clades

| Clade | R203K/G204R Counts |
|---|---|
| 19A | 22 |
| 19B | 4 |
| 20A | 523 |
| 20A.EU2 | 30 |
| 20B | 114,487 |
| 20C | 12 |
| 20D | 5,671 |
| 20E (EU1) | 15 |
| 20F | 12,593 |
| 20G | 2 |
| 20H/501Y.V2 | 5 |
| 20I/501Y.V1 | 98,552 |
| 20J/501Y.V3 | 245 |

Plasmid and cloning. The pLVX-EFlalpha-SARS-CoV-2-N-2×Strep-IRES-Puro was a gift from Nevan Krogan (Addgene plasmid #141391; RRID:Addgene_141391)³⁹. The three consecutive SNPs (G28881A, G28882A, G28883C), corresponding to N protein mutation sites R203K and G204R, were introduced by megaprime PCR mutagenesis using the primers listed in Table 1.

Cell culture and transfection. HEK293T (ATCC; CRL-3216) cells were grown in Dulbecco's modified Eagle's medium (DMEM) (4.5 g/l d-glucose and Glutamax, 1 mM sodium pyruvate) (GIBCO) and 10% fetal bovine serum (FBS; GIBCO) with penicillin-streptomycin supplement, according to standard protocols (culture condition 37° C. and 5% CO₂). Calu-3 (ATCC HTB-55) cells were grown in DMEM (1.0 g/l d-glucose, 2 mM L-glutamine, 1 mM sodium pyruvate) with addition of 1% non-essential amino acids, 1500 mg/L sodium bicarbonate, and 10% fetal bovine serum (FBS; GIBCO). Transfection of ten million cells per 15-cm dish with 2λStrep-tagged N plasmid (20 ug/transfection) was performed using lipofectamine-2000 according to standard protocol. For Calu-3 cells, double transfection was conducted; the first transfection was done on the day of cell splitting and the second after 24 h of culture.

Affinity purification and on-bead digestion. Cell lysis and affinity purification with MagStrep beads (IBA Lifesciences) were manually performed according to the published protocol³⁹ with minor modifications. Briefly, after transfection (48 h) cells were collected with 10 mM EDTA in 1×PBS and washed twice with cold PBS (samples used for phosphorylation analysis were collected and washed in the presence of phosphatase inhibitor cocktails). The cell pellets were stored at −80° C. Cells were lysed in lysis buffer (50 mM Tris-HCl, pH 7.4, 150 mM NaCl, 1 mM EDTA, 0.5% NP40, supplemented with protease and phosphatase inhibitor cock-tails) for 30 min while rotating at 4° C. and then centrifuge at high speed to collect the supernatant. The cell lysate was incubated with prewashed MagStrep beads (30 μl per reaction) for 3 h at 4° C. The beads were then washed four times with wash buffer (50 mM Tris-HCl, pH 7.4, 150 mM NaCl, 1 mM EDTA, 0.05% NP40 supplemented with protease and phosphatase inhibitor cocktails) and then proceed with on-bead digestion. The on-bead digestion was carried out as described before³⁹. Briefly, after the final wash the beads were washed once with exchange buffer (50 mM Tris-HCl PH 7.5, 100 mM NaCl). Bead-bound proteins were alkylated with 3 mM iodoacetamide in the dark for 45 min and then quenched with 3 mM DTT for 15 min. Digestion was performed using 1 μl trypsin (1 μg/μl, Pro-mega) overnight with shaking (1000 rpm) and the peptides were then purified. For affinity confirmation, bound proteins were eluted using buffer BXT (IBA Life-sciences) and after running on SDS-PAGE were subjected to silver staining and western-blot using anti-strep-II antibody (ab76949) (dilution used 1:1000). To purify clean 2×Strep-tagged N protein (mutant and control), stringent washing and double elution strategy were applied.

MS analysis using Orbitrap Fusion Lumos. The MS analysis was performed as described previously^{82,83} with slight modifications. Briefly, approximately 0.5 μg of peptide mixture in 0.10% formic acid (FA) was injected into a nanotrap (PepMap 100, C18, 75 μm×20 mm, 3 μm particle size) and desalted for 5 min with 0.1% FA in water at a flow rate of 5 μl/min. They were then eluted and analyzed using an Orbitrap Fusion mass spectrometer (MS) (Lumos, Thermo Fisher Scientific) cou-pled with an UltiMate™ 3000 UHPLC (Thermo Scientific). The peptides were separated by an EasySpray C18 column (50 cm×75 μm ID, PepMap C18, 2 μm particles, 100 Å pore size, Thermo Scientific) with a 75-min gradient at constant 300 nL/min, at 40° C. The electrospray potential was set at 1.9 kV, and the ion transfer tube temperature was set at 270° C. A full MS scan with a mass range of 350-1500 m/z was acquired in the Orbitrap at a resolution of 60,000 (at 400 m/z) using the profile mode, a maximum ion accumulation time of 50 ms, and a target value of 2e5. The most intense ions that were above a 2e4 threshold and carried multiple positive charges (2-6) were selected for fragmentation (MS/MS) via higher energy collision dissociation (HCD) with normalized collision energy at 30% within the 2 s cycling time. The dynamic exclusion was 30 s. The MS2 was acquired with data type as centroid at a resolution of 30,000. Protein identification analysis from the raw mass spectrometry data was performed using the Maxquant software (v1.5.3.30)³⁸ as described⁸².

For phosphorylated peptides, a Maxquant label-free quantification (LFQ)³⁸ was used. The analysis and quantification of phosphorylated peptides were performed according to published protocol⁸⁴.

Analysis of differential interaction. First, the identified protein group data were corrected for non-specific background binding by removing all proteins detected in mock control (cells transfected with the plasmid vector without N gene) affinity mass spectrometry. The normalized LFQ data were processed for statistical analysis on the LFQ-Analyst a web-based tool to performed pair-wise comparison between mutant and control N protein AP-MS data. The significant differentially changed proteins between mutant and control conditions were identified. The threshold cutoff of adjusted p-value<=0.05, and Log fold-change>=1 were used. Among the replicates, outliers were removed based on correlation and PCA analysis. The GO-enrichment analysis was performed on the LFQ-Analyst⁸⁵.

BS3 cross-linking. Bis(sulfosuccinimidyl) suberate (BS3, Thermo Scientific Pierce) was used for cross-linking of control and mutant N protein to analyse the oligo-merization properties. The experiment was performed as reported previously⁸⁶. Briefly, 2×Strep-tagged N protein (mutant and control) was purified as mentioned above. The purified N protein (mutant and control) were cross-linked using 2 mM Bis(sulfosuccinimidyl) suberate (BS3) for 30 min at room temperature. The control and cross-linked forms of N proteins (mutant and control) were separated on SDS-PAGE, subjected to silver staining and densitometry analysis of bands. GraphPad Prism (v9.1.1) was used for analysis and graph generation.

In vitro protein-RNA interaction (RIP) assay. The in vitro interaction assay was performed using purified 2×Strep-tagged N protein (mutant and control) and total isolated RNA from patient swabs as mentioned above in the RNA isolation section. The 2×Strep-tagged (in bead-bound condition) N protein (mutant and control) was incubated with total RNA in reaction buffer (50 mM Tris HCl pH7.5, 150 mM KCl, 0.1 mM EDTA, 1 mM DTT, 5% Glycerol, 0.02% NP40, 1 mM PMSF, 40 U RNase OUT™). After shaking incubation for 45 min, N proteins (mutant and control) were pulldown using MagStrep beads on a magnetic separator with four washes (using the same reaction buffer). After the final wash, the RNA was isolated by adding Trizol using the Zymo direct-zol method. The isolated RNAs were analyzed by RT-qPCR using specific viral N gene (N1 and N2), E gene, S gene, and ORF1ab region primers (Table 2). GraphPad Prism (v9.1.1) was used for analysis and graph generation.

RNA-sequencing and differential gene expression analysis. Calu-3 cells were transfected with plasmids expressing the full-length N-control and N-mutant protein along with mock control. After 48-h cells were harvested in Trizol and total RNA was isolated using Zymo-RNA Direct-Zol kit (Zymo, USA) according to the manufacture's instruction. The concentration of RNA was measured by Qubit (Invitrogen), and RNA integrity was determined by Bioanalyzer 2100 system (Agilent Technologies, CA, USA). The RNA was then subjected to library pre-paration using Ribozero-plus kit (Illumina). The libraries were sequenced on NovaSeq 6000 platform (Illumina, USA) with 150 bp paired-end reads.

The raw reads from Calu-3 RNA-sequencing were processed and trimmed using trimmomatic⁶³ and mapped to annotated ENSEMBL transcripts from the human genome (hg19)^{87,88} using kallisto (v0.43.1)⁸⁹. Differential expression analysis was performed after normalization using EdgeR integrated in the NetworkAnalyst90. GO biological process and pathway enrichment analyses on differentially expressed genes were performed using NetworkAnalyst90.

### Results

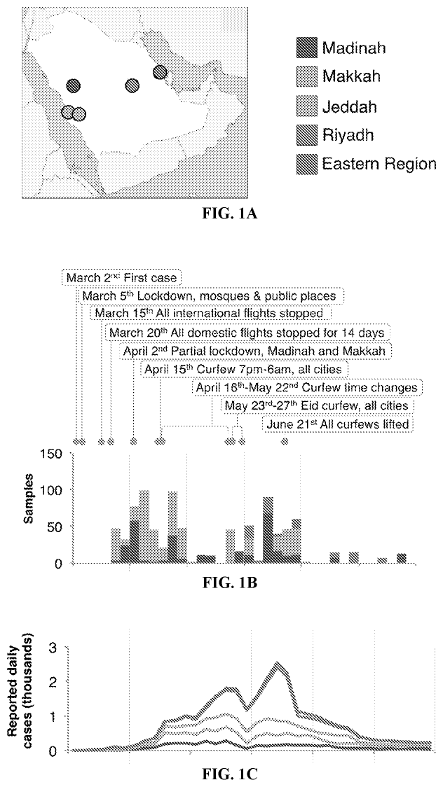

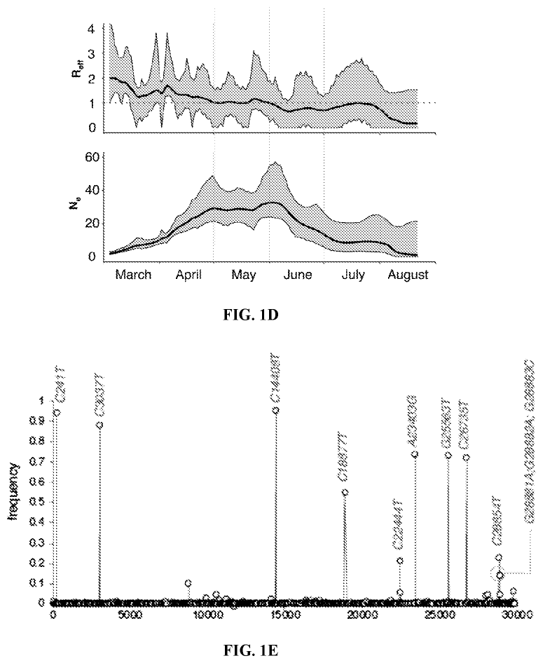

In this study, 892 SARS-CoV-2 genomes from nasopharyngeal swab samples of patients from the four main cities, Jeddah, Makkah, Madinah, and Riyadh, as well as a small number of patients from the Eastern region of Saudi Arabia were sequenced (FIG. 1A). The genomes were analyzed to investigate the nucleotide changes and multiple mutation events that represent the first 6 months of the locally circulating pandemic lineages of the SARS-CoV-2 in Saudi Arabia and searched for potential association of polymorphic sites in the genome with available hospital records including severe disease and case fatality rates among the Covid-19 patients (FIGS. 1B-1C). Phylogenetic analysis was performed to visualize the genetic diversity of SARS-CoV-2 and the nature of transmission lineages during March-August 2020. A snapshot of the genomic variation landscapes of the SARS-CoV-2 lineages in this study population is presented here and linked specific set of mutation events in the N gene to the disease outcome in a diverse population of Covid-19 patients in Saudi Arabia. Further support for this functional linkage was also found in global data sets. Finally experiments show the functional impact of these mutations in the N protein on the virus' life cycle and its interaction with the host.

### Phylogenetic Analysis of Assembled Genomes

SARS-CoV-2 genomes from 892 patient samples were sequenced and assembled. This group includes two patients that had tested negative for COVID-19 at the hospitals, and 144 patients that were placed in quarantine and had either mild symptoms or were asymptomatic. The remaining patients were all hospitalized. Data on comorbidities were available for 689 patients with diabetes (39%) and hypertension (35%) being the most abundant. Patient outcome data was available for 850 samples, and 199 patients (23%) died during hospitalization).

From the 892 assembled viral genomes collected over a period of 6 months, a total of 836 single-nucleotide polymorphisms (SNPs) were identified, compared to the Wuhan SARS-CoV-2 reference (GenBank accession: NC_045512) (FIG. 1E). Together with all sequences from Saudi Arabia available on GISAID on Dec. 31, 2020, the assembled sequences were used to construct effective population size and growth rate estimates of SARS-CoV2 over the course of the first wave of the epidemic. The skygrowth model (FIG. 1D) shows a downward trend in the effective reproduction number (R) over time with the introduction and maintenance of non-pharmaceutical interventions. Following the lifting of restrictions towards the end of June, the model estimates that R remained below or at 1 to the end of the period covered by the genetic data presented in this paper. The effective population size (Ne) represents the relative diversity of the sequences collected in Saudi Arabia over the course of the outbreak, suggesting that diversity peaked at the start of June, ahead of the peak in cases seen in the country in mid-June (FIG. 1D). This aligns with the peak number of reported cases in the regions, which contribute the most to the included genetic sequences (Riyadh, June 16th; Jeddah, May 30th; Makkah, June 6th; Madinah May 6th).

A phylogenetic analysis revealed that samples from Saudi Arabia represent 5 major Nextstrain clades¹⁰, 19A, 19B, 20A, 20B, and 20C. Time-scaled phylogenies dates of importation events were estimated for each clade: 19A dominating in early outbreak (7), 19B dominating in early outbreak (42), 20A European outbreak in March, spreading globally (761), 20B distinct subclade of 20A, emerged early 2020 (139), and 20C distinct subclade of 20A, emerged early 2020 (3). The majority of importations for all clades were inferred to have occurred early in the outbreak, primarily in March and early April (FIG. 2). Based on available genetic data, clade 19B was likely the first introduced into Saudi Arabia with the most likely importation date for this clade falling in February. The country of origin behind importation events was further estimated, suggesting multiple imports from countries across Asia, Europe, Asia, Oceania, America and Africa. A detailed maximum-likelihood tree of Saudi Arabia genomes highlights a rapidly diverging subgroup largely consisting of clade 20B virus samples, predominately from Jeddah, with high incidences of ICU hospitalizations and subsequently fatal outcomes.

A Mutant Form of the Nucleocapsid (N) Protein Associated with Patient Mortality

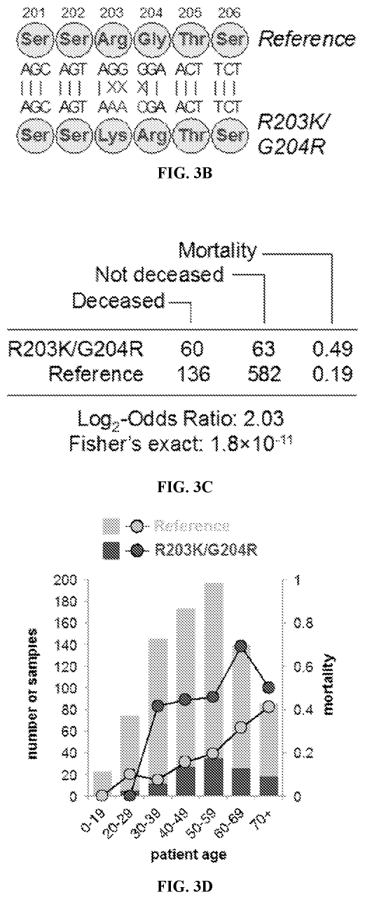

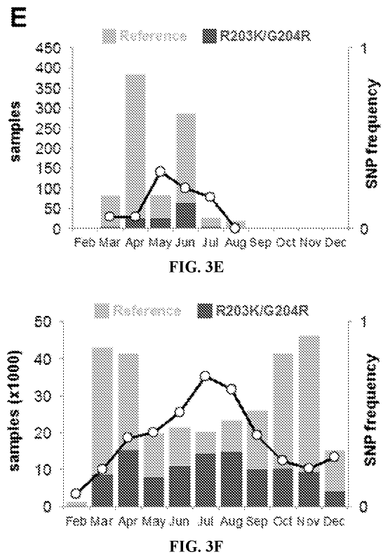

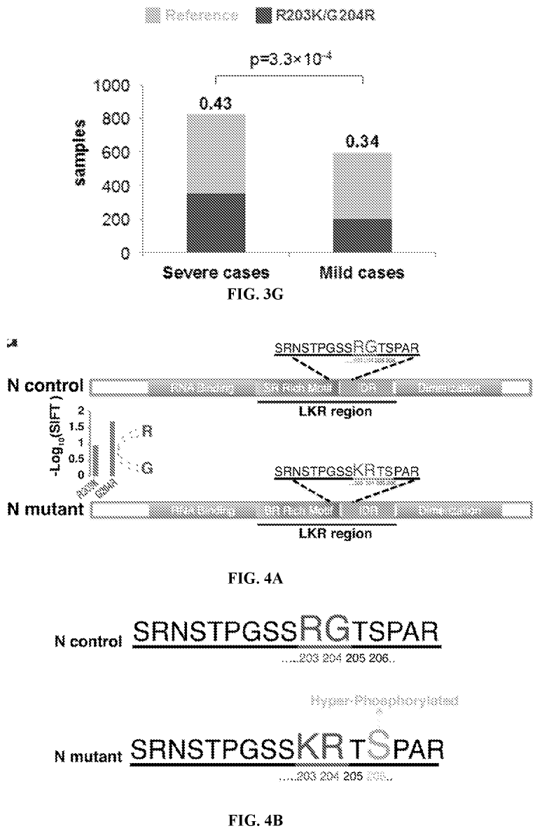

A genome-wide association study between SARS-CoV-2 SNPs and patient mortality highlighted these three consecutive SNPs (G28881A, G28882A, G28883C) resulting in the previously reported Nucleocapsid (N) protein mutations R203K and G204R¹³ (FIGS. 3A and 3B). Of the 892 assembled genomes, 882 (98.9%) genomes either have the three reference alleles, GGG, or the three mutant alleles, AAC, at positions 28,881-28,883. This is similarly found in global samples deposited in GIASID in 2020, where 98.6% of samples either have reference alleles or all three SNPs, with this mutant triplet being the fifth most abundant across all samples.

No other SNPs show similar levels of association with mortality (FIG. 3A), and consistent with this, no other SNPs were identified co-occurring with the R203K/G204R SNPs (FIGS. 5A-5B).

The mortality rate for samples with the R203K/G204R SNPs is 0.49 compared to 0.19 for samples without the SNPs (FIG. 3C). The significantly increased mortality was observed for both male and female patients separately (data not shown). Of the 124 samples with the R203K/G204R SNPs (one sample did not have patient outcome information and is excluded from FIG. 3C), 101 samples are from Jeddah. Mortality among Jeddah patients (0.40) is vastly higher than among patients from all other cities (0.16) (data not shown). The observed mortality rate among samples R203K/G204R SNPs is higher when only considering samples from Jeddah (0.55 versus 0.30) and samples from outside Jeddah (0.23 versus 0.16), although this difference is only significant for the former. To further assess the association between with the R203K/G204R SNPs and mortality, genotype information at positions 28,881-28,883 from samples for which no complete genome could be assembled and from samples genotyped by Sanger sequencing were included. The addition of 304 such samples confirmed the association between these SNPs and patient mortality (Fisher's exact, p=4.7×10⁻¹⁰; log 2 odds ratio: 1.58). No apparent difference between patient ages is observed between samples with the R203K/G204R SNPs and the remaining samples, and the excess mortality is generally observed across the entire range of patient ages (FIG. 3D). The association between R203K/G204R SNPs and mortality is still observed after correcting for comorbidities. Consistent with their association to disease severity, the R203K/G204R SNPs are significantly less prevalent in samples from patients in quarantine compared to hospitalized patients, and correspondingly more abundant in samples from patients submitted to ICU.

A time-scaled phylogenetic approach suggest that the R203K/G204R SNPs originated late January 2020, although the earliest sampled genome with the SNPs is only available from February 23rd. The phylogenetic distribution of viral genomes harboring the R203K/G204R SNPs implies that the SNPs may have originated independently at least twice during the pandemic.

Within the sampling window of the present studies a transient increase in the frequency of R203K/G204R SNPs was observed (FIG. 3E) in accordance with earlier observations^{13,16}. This is further underlined by global data showing a clear increase of R203K/G204R in the first half of 2020 reaching a frequency of 0.75, followed by a similar decrease in the second half of the year (FIG. 3F). Although R203K/G204R frequencies vary, the overall trend is generally observed across all continents.

Subsequent studies sought to test the association between mortality and the R203K/G204R SNPs on a global scale and collected 17,261 non-Saudi samples with patient metadata from GISAID (Dec. 31, 2020). The reporting format is highly non-standardized, therefore, a manually curated list of terms reflecting two different disease outcomes: severe cases (deceased patients, critically ill patients, and cases submitted to ICU) and mild cases. This reduced the available samples to 1,419. Similar to our observations from Saudi samples, studies show that the samples from severe cases display a significantly higher frequency of R203K/G204R SNPs compared to mild cases (FIG. 3G). The available metadata are far from ideal for this type of analysis and numbers should be interpreted with great caution, but nevertheless this observation is considered in independent support of our findings among samples from Saudi Arabia.

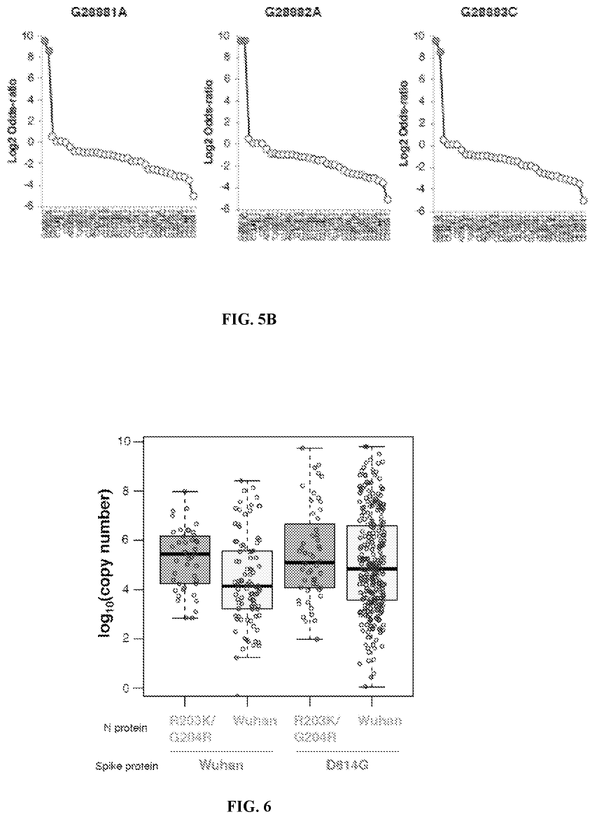

The cycle threshold (Ct) values obtained through quantitative PCRs can be used as a proxy for viral load and even a predictor of clinical outcome¹⁷. Earlier, a non-synonymous SNP in the Spike (S) protein, D614G, was found to be associated with higher viral load¹⁸⁻²⁰. A significantly higher viral copy number was found in samples with the either D614G or R203K/G204R SNPs, as well as samples with all the SNPs, than in samples with the Wuhan reference alleles (FIG. 6).

### N Mutant Protein has High Oligomerization Potential and RNA Binding Affinity

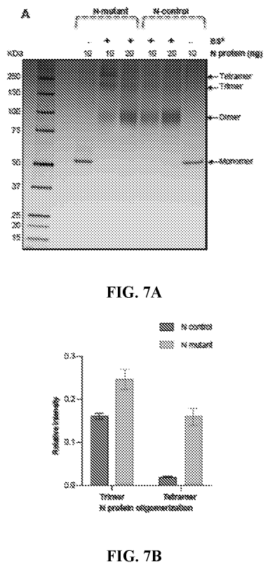

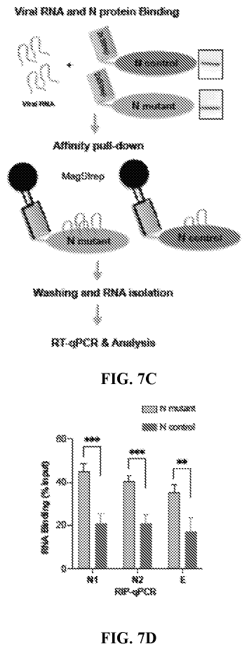

The SARS-CoV-2 N protein binds the viral RNA genome and is central to viral replication. Protein structure predictions have shown that the R203K/G204R mutations result in significantly changes in protein structure¹⁶, theoretically destabilizing the N structure²², and potentially enhancing the protein's ability to bind RNA and alter its response to serine phosphorylation events²³. The R203K/G204R mutations in the SARS-CoV-2 N protein are within the linkage region (LKR) containing the serine/arginine-rich motif (SR-rich motif) (FIG. 4A), known to be involved in the oligomerization of N proteins^{24,25}. Protein cross-linking shows that N mutant protein (with the R203K/G204R mutations) has higher oligomerization potential compared to the control N protein (without the changed amino acids) at low protein concentration (FIGS. 7A-7B). However, both mutant and control N protein formed dimers with similar efficiency (FIG. 7A).

Given that the oligomerization of N protein acts as a platform for viral RNA interactions²⁶, further studies sought to examine the binding affinity of mutant and control N protein with viral RNA isolated from COVID-19 patient swabs. The RNA-binding activity of mutant and control N proteins was examined by pulled-down viral RNA through in vitro RIP assay (FIG. 7C), and the data revealed that mutant N protein enriched significantly higher level of viral RNA compared to control protein (FIG. 7D). This indicates a strong binding capability of mutant N proteins with viral RNA, which could potentially impact the essential roles of N protein at various stages of viral life cycle and its interaction with the host.

The R203K/G204R Mutations in the N Protein Affect its Interaction with Host Proteins

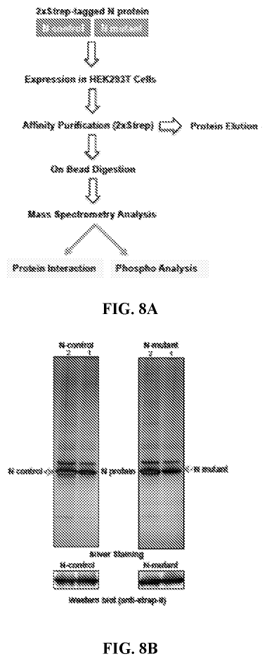

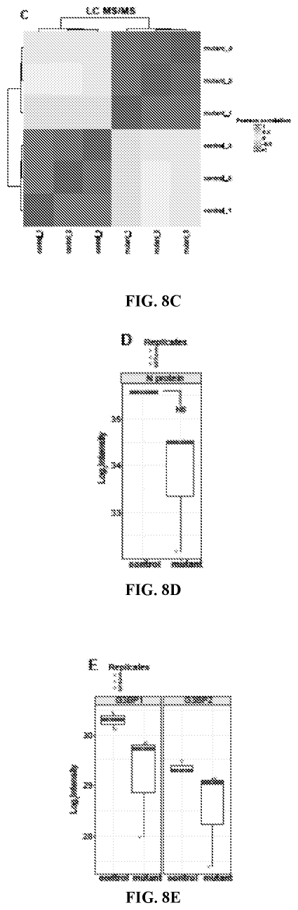

According to the SIFT tool²¹, a substitution at position 204 from G to R in the N protein is predicted to affect functional features (FIG. 4A). Therefore, additional studies investigated how the two amino acids substitution (R203K and G204R) in the N protein impact its functional interaction with the host that could modulate viral pathogenesis and rewiring of host cell pathways and processes. HEK-293T cells (3 biological replicates,) were used for affinity-purification followed by mass spectrometry analysis (AP-MS) to identify host proteins associated with control and mutant N protein (FIGS. 8A-8E). 48 human proteins were that displayed significant (adjusted p-value≤0.05, and Log fold change≥1) differential interactions with mutant and control N protein (data not shown). Among these, 43 proteins showed increased interaction and 5 proteins showed decreased interaction with the N mutant. The total level of mutant and control N protein remained unchanged (FIG. 8D). These studies indicated that the stress granule proteins G3BP1 and G3BP2 showed comparable interaction in mutant and control conditions (FIG. 8E). Among the group with increased interaction, many proteins associated with TOR and other signaling pathways (such as AKT1S1, GSK3A, and PIN1), proteins associated with the viral process, viral transcription, and negative regulation of RNA nuclear export (NUP98 and NUP153), and proteins involved in apoptotic and cell death processes (PAWR, ACIN1, and PDCD5) were identified. Proteins in the mutant condition that are linked with the immune system processes (PTMS), kinase activity (GCN1), and translation (e.g., MRPS36) were also identified. In the group with decreased interaction, SNIP1 (NF-kappaB signaling), TMA16 (translation), and CSNK2B (casein kinase II). Gene ontology analysis showed that the most enriched biological processes are associated with negative regulation of tRNA and ribosomal subunit export from the nucleus. This finding suggests that the mutant virus can efficiently inhibit and hijack the host translation to facilitate viral replication and pathogenesis. In reactome enrichment analysis, pathways associated with the sumoylation of host proteins and antiviral mechanisms were identified. Further, many viruses can manipulate the host sumoylation process to enhance viral survival and pathogenesis²⁸.

### Serine 206 (S206) Displays Hyper-Phosphorylation in the Mutant N Protein

In SARS-CoV, it has been shown that phosphorylation of N protein is more prevalent during viral transcription and replication²⁹ and inhibition of phosphorylation diminishes viral titer and cytopathogenic effects³⁰. Recent elegant studies elaborated the role of N protein phosphorylation in modulating RNA binding and phase separation in SARS-CoV-2^{26,31-33} Thus, phosphorylation of N protein in the LKR region is critical for regulating both viral genome processing (transcription and replication) and nucleocapsid assembly^{26,31}. To further understand the functional relevance of KR mutation in the N protein, phosphoproteomic analysis were performed in control and mutant conditions. The studies consistently found that the serine 206 (S206) site, which is next to the KR mutation site (FIG. 4B), is highly phosphorylated, specifically in the mutant N protein (FIG. 4F). Notably, the total amount of N protein (FIG. 8D) and the phosphorylation at serine 2 (S2) and other sites (S79, S176, and S180) in the LKR region did not change between mutant and control conditions (FIG. 4C).

### Discussion

From 892 samples collected across the country over the course of approximately 6 months the dynamics of transmission and diversity of SARS-CoV-2 in Saudi Arabia was analyzed. The lineage analysis of assembled genomes highlights the repeated influx of SARS-CoV-2 lineages into the Kingdom through international travels.

The detailed patient data allowed the detection of three SNPs—underlying the N protein R203K and G204R mutations—associated with significantly increased mortality rates. In publicly available global samples with relevant patient information these SNPs were found to be similarly associated with increased mortality. These findings thus strongly suggest that the R203K and G204R mutations in the N protein play a role in the severity of the COVID-19 disease not only in Saudi Arabia but also supported by sparsely available global datasets with relevant clinical outcome and mortality data.

The trade-off model for virulence evolution—although challenged by certain empirical observations^{34,35}—implies that higher virulence comes at a cost for the virus reproduction rate if not counteracted by changes in the recovery or the transmission rates^{36,37} In this respect, the decrease in the frequency of R203K and G204R mutations during the late half of 2020 (FIG. 3F), suggest that the associated mortality of the mutations negatively affects their associated reproduction rate. This is in contrast to the Spike protein mutation D614G, the B.1.1.7 lineage, and the 501.V2 variant that—although more transmissible—are apparently not associated with higher virulence and subsequently increase in the global populationis^{18,20,38,39}.

The N protein of SARS-CoV-2, a highly abundant structural protein within the infected cells, serves multiple functions during viral infection, which besides RNA binding, oligomerization, and genome packaging, playing essential roles in viral transcription, replication, and translation^{40,41}. Also, the N protein can evade immune response and perturbs other host cellular processes such as translation, cell cycle, TGFβ signaling, and induction of apoptosis⁴² to enhance virus survival. The critical functional regulatory hub within the N protein is a conserved serine-arginine (SR) rich-linker region (LKR), which is involved in RNA and protein binding⁴³, oligomerization^{24,25}, and phospho-regulation^{26,31}.

The data shows that the mutant N protein containing R203K and G204R changes has higher oligomerization and stronger viral RNA binding ability, suggesting a potential link of these mutations with efficient viral genome packaging. The R203K and G204R mutations are in close proximity to the recently reported RNA-mediated phase separation domain (aa 210-246) 33 that is involved in viral RNA packaging through phase separation. This domain was thought to enhance phase-separation also through protein-protein interactions³³.

Moreover, the functional activities of the N protein at different stages of viral life cycle are regulated by phosphorylation-dependent physiochemical changes in the LKR region³¹. Although all individual phosphorylation sites may not be functionally important^{23,44} the specific enhancement of phosphorylation at serine 206 in the mutant N protein shown in this study suggests a functional significance. The serine 206 can form a phosphorylation-dependent binding site for protein 14-3-3, involved in cell cycle regulatory pathways regulating human and virus protein expression 45 Multiple lines of evidence show that N protein phosphorylation is critical for its dynamic localization and function at replication-transcription complexes (RTC), where it promotes viral RNA transcription and translation by recruiting cellular factors^{29-31,46-49}. The enrichment of glycogen synthase kinase 3 A (GSK3A) with the mutant N protein, could specifically phosphorylate serine 206 in the R203K/G204R mutation background. GSK3 was shown to be a key regulator of SARS-CoV replication due to its ability to phosphorylate N protein³⁰. Phosphorylation of serine 206 acts as priming site for initiating a cascade of GSK-3 phosphorylation events^{30,31}. Also, GSK3 inhibition dramatically reduces the production of viral particles and the cytopathic effect in SARS-CoV-infected cells³⁰.

In conclusion, the results presented herein highlight the influence of the R203K/G204R mutations on the essential properties and phosphorylation status of SARS-CoV-2 N protein that lead to increased efficacy of viral infection, potentially underlying the observed rise in mortality observed during these genome analysis.

### REFERENCES

1 Organization, W. H. Coronavirus disease (COVID-19) Weekly Epidemiological Update and Weekly Operational Update, <www.who.int/emergencies/diseases/novel-coronavirus-2019/situation-reports>(2020).
2 Dong, E., Du, H. & Gardner, L. An interactive web-based dashboard to track COVID-19 in real time. Lancet Infect Dis 20, 533-534, doi:10.1016/S1473-3099(20)30120-1 (2020).
3 Center, J. H. U. M. C. R. COVID-19 Dashboard, <https://coronavirus.jhu.edu/map.html>(2020).
4 Ebrahim, et al., Lancet 395, e48, doi:10.1016/S0140-6736(20)30466-9 (2020).
5 Memish, et al., Lancet Infect Dis, doi: 10.1016/S1473-3099(20)30425-4 (2020).
6 Tuite, et al., Ann Intern Med 172, 699-701, doi:10.7326/M20-0696 (2020).
7 News, A. Saudi Arabia announcesfirst case of coronavirus, <https://www.arabnews.com/node/1635781/saudi-arabia>(2020).
8 Gussow, et al., Proceedings of the National Academy of Sciences of the United States of America 117, 15193-15199, doi:10.1073/pnas.2008176117 (2020).
9 Lu, et al., Cell 181, 997-1003 e1009, doi:10.1016/j.cell.2020.04.023 (2020).
Hadfield, et al., Bioinformatics 34, 4121-4123, doi:10.1093/bioinformatics/bty407 (2018).
11 Rambaut, et al., Nat Microbiol 5, 1403-1407, doi:10.1038/s41564-020-0770-5 (2020).
12 Boni, et al., Nat Microbiol 5, 1408-1417, doi:10.1038/s41564-020-0771-4 (2020).
13 Leary, et al., bioRxiv, 2020.2004.2010.029454, doi:10.1101/2020.04.10.029454 (2020).
14 Toyoshima, et al., J Hum Genet 65, 1075-1082, doi:10.1038/s10038-020-0808-9 (2020).
15 Singh, et al., bioRxiv, 2020.2009.2017.301614, doi:10.1101/2020.09.17.301614 (2020).
16 Wu, et al., J Med Virol, doi: 10.1002/jmv.26597 (2020).
17 Rao, et al., Infect Dis Ther 9, 573-586, doi:10.1007/s40121-020-00324-3 (2020).
18 Korber, et al., Cell 182, 812-827 e819, doi:10.1016/j.cell.2020.06.043 (2020).
19 Lorenzo-Redondo, et al., medRxiv, 2020.2005.2019.20107144, doi:10.1101/2020.05.19.20107144 (2020).
20 Volz, E. et al., Cell 184, 64-75 ell, doi:10.1016/j.cell.2020.11.020 (2021).
21 Ng, et al., Nucleic Acids Res 31, 3812-3814, doi:10.1093/nar/gkg509 (2003).
22 Rahman, et al., J Med Virol, doi: 10.1002/jmv.26626 (2020).
23 Guan, et al., Int J Infect Dis 100, 216-223, doi:10.1016/j.ijid.2020.08.052 (2020).
24 He, et al., Biochem Bioph Res Co 316, 476-483, doi: 10.1016/j.bbrc.2004.02.074 (2004).
Chang, et al., Plos One 8, doi:ARTN e65045 10.1371/journal.pone.0065045 (2013).
26 Chao et al., BioRxiv, doi:https://doi.org/10.1101/2020.11.30.404905 (2020).
27 Gordon, et al., Nature 583, 459-+, doi:10.1038/s41586-020-2286-9 (2020).
28 Lowrey, et al., Cell Commun Signal 15, doi:ARTN 27 10.1186/s12964-017-0183-0 (2017).
29 Wu, et al., Cell Host Microbe 16, 462-472, doi:10.1016/j.chom.2014.09.009 (2014).
Wu, et al., J Biol Chem 284, 5229-5239, doi: 10.1074/jbc.M805747200 (2009).
31 Carlson, et al., Mol Cell 80, 1092-+, doi:10.1016/j.molcel.2020.11.025 (2020).
32 Savastano, et al., Nat Commun 11, doi:ARTN 6041 10.1038/s41467-020-19843-1 (2020).
33 Lu, et al., Nature communications 12, 502, doi:10.1038/s41467-020-20768-y (2021).
34 Geoghegan, et al., Nat Rev Genet 19, 756-769, doi:10.1038/s41576-018-0055-5 (2018).
35 Alizon, et al., J Evol Biol 22, 245-259, doi:10.1111/j.1420-9101.2008.01658.x (2009).
36 Anderson, et al., Parasitology 85 (Pt 2), 411-426, doi:10.1017/s0031182000055360 (1982).
37 Anderson, et al., Nature 280, 361-367, doi:10.1038/280361a0 (1979).
38 Arif, et al., Infect Control Hosp Epidemiol, 1-2, doi:10.1017/ice.2020.1434 (2021).
39 Grubaugh, et al., Cell 182, 794-795, doi:10.1016/j.cell.2020.06.040 (2020).
40 McBride, et al., Viruses 6, 2991-3018, doi:10.3390/v6082991 (2014).
41 Chang, et al., Antiviral Res 103, 39-50, doi:10.1016/j.antiviral.2013.12.009 (2014).
42 Lal, M. S. a. S. K. in Molecular Biology of the SARS-Coronavirus (ed Sunil K. Lal) 129-151 (2009).
43 Wegener, et al., Adv Exp Med Biol 1203, 83-112, doi:10.1007/978-3-030-31434-7_3 (2019).
44 Bouhaddou, et al., Cell 182, 685-712 e619, doi: 10.1016/j.cell.2020.06.034 (2020).
Nathan, et al., Viruses 12, doi: 10.3390/v12040436 (2020).
46 Verheije, et al., J Virol 84, 11575-11579, doi:10.1128/Jvi.00569-10 (2010).
47 Chen, et al., J Virol 79, 1164-1179, doi:10.1128/Jvi.79.2.1164-1179.2005 (2005).
48 Peng, et al., Febs J 275, 4152-4163, doi:10.1111/j.1742-4658.2008.06564.x (2008).
49 V'kovski, et al., Elife 8, doi:ARTN e42037 10.7554/eLife.42037 (2019).

We claim:

1. A capture protein comprising
i) a nucleocapsid (N) protein derived from SARS-CoV-2 virus, or a functional fragment or variant thereof, wherein the N protein or variant thereof comprises an R203K/G204R mutation
ii) one or more capture tags,
wherein the one or more capture tags are covalently conjugated to the capture protein.

2. The capture protein of claim 1, wherein the nucleocapsid protein has the amino acid sequence of SEQ ID Nos. 1-4.

3. The capture protein of claim 2, wherein the nucleocapsid protein has a lysine (K) at the amino acid position 203, or an arginine (R) at the amino acid position 204, or both.

4. The capture protein of claim 1, wherein the functional fragment comprises between about 50 and about 419 contiguous amino acids of the nucleocapsid protein, wherein the functional fragment is sufficient to maintain the biological function of binding to viral RNA.

5. The capture protein of claim 1, wherein the variant comprises at least 65%, 70%, 75%, 80%, 85%, 90%, 95%, 99% or 100% sequence identity to SEQ ID NOs:1, 2, 3, or 4.

6. The capture protein of claim 1, wherein the capture tag is selected from the group consisting of hexa-histidine tag, FLAG tag, Strep II tag, streptavidin-binding peptide (SBP) tag, calmodulin-binding peptide (CBP), glutathione S-transferase (GST), maltose-binding protein (MBP), S-tag, the hemagglutinin (HA) tag, and c-Myc tag.

7. The capture protein of claim 1, further comprising a support matrix.

8. The capture protein of claim 7, wherein the support matrix is a solid phase selected from the group consisting of glass plates, microtiter well plates, magnetic beads, and silicon wafers.

9. The capture protein of claim 8, wherein the solid phase is magnetic beads.

10. The capture protein of claim 8, wherein the magnetic beads are spherical beads having a diameter of about 1 μm to about 10 μm, inclusive, or about 2 μm and about 8 μm, inclusive, or about 3 μm and about 6 μm.

11. The capture protein of claim 1, further comprising iii) one or more RNAs bound to the capture protein.

12. The capture protein of claim 11, wherein the one or more RNAs are viral RNAs derived from an RNA virus, optionally, wherein the RNA virus is a coronavirus, optionally, wherein the coronavirus is SARS-CoV-2.

13. (canceled)

14. (canceled)

15. (canceled)

16. (canceled)

17. (canceled)

18. A method of enriching and/or isolating viral RNAs comprising
(a) contacting a sample of interest with the capture protein of claim 1, in a binding buffer.

19. The method of claim 18, wherein the capture protein is immobilized to a solid phase selected from the group consisting of glass plates, microtiter well plates, magnetic beads, and silicon wafers.

20. The method of claim 19, wherein the capture protein is immobilized to magnetic beads.

21. The method of any claim 18, wherein the binding buffer has a pH of about 7 to about 8, preferably 7.5, and wherein the binding buffer comprises one or more RNase inhibitors.

22. The method of claim 18, further comprising the step of
(b) removing supernatant after step (a).

23. The method of claim 18, further comprising
(c) one or more washing steps to remove unbound and/or non-specific bound molecules.

24. The method of claim 18, further comprising
(d) isolating the capture protein complexed with viral RNA.

25. The method of claim 18, further comprising
(e) eluting isolated viral RNA from the capture protein using an appropriate buffer.

26. The method of claim 18, further comprising
(f) quantitating the isolated viral RNA, optionally, wherein viral RNA is derived from an RNA virus.

27. The method of claim 25, wherein the quantitating the isolated viral RNA includes the step of amplifying one or more genomic segments of the isolated viral RNA.

28. The method of claim 27, wherein the amplification comprises a reverse transcription quantitative polymerase chain reaction (RT-qPCR).

29. (canceled)

30. The method of claim 29, wherein the RNA virus is a coronavirus, optionally, wherein the coronavirus is SARS-CoV-2.

31. (canceled)

32. The method of claim 18, wherein the sample of interest is an environmental sample or a biological sample; and/or wherein the sample of interest contains 10 or more copies of the SARS-CoV-2 genomes.

33. The method of claim 32, wherein the biological sample is a bodily fluid of a subject, the bodily fluid selected from the group consisting of mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, and whole blood.

34. The methods of claim 33, wherein the method further comprises a step of obtaining the biological sample from the subject, optionally, wherein the biological sample is obtained from a nasopharyngeal swab, a nasopharyngeal aspirate, sputa/deep throat saliva, or a throat swab of the subject.

35. (canceled)

36. The method of claim 33, wherein the subject is selected from the group consisting of a subject who has one or more symptoms of COVID-19, an asymptomatic subject who is at increased risk of being infected with SARS-CoV-2 virus, a subject who has received a vaccine against infection with SARS-CoV-2 virus, and a deceased subject.

37. (canceled)
